(19) United States
(12) Patent Application Publication
(10) Pub. No.: US 2022/0280557 A1
(43) Pub. Date: Sep. 8, 2022

(54) Metabolic Reprogramming of Immune Cells for the Treatment or Prevention of Diseases and Disorders

(71) Applicant: Arizona Board of Regents on behalf of Arizona State University, Scottsdale, AR (US)

(72) Inventors: Abhinav Acharya, Tempe, AZ (US); Joslyn Mangal, Tempe, AZ (US)

(21) Appl. No.: 17/636,209

(22) PCT Filed: Sep. 11, 2020
(86) PCT No.: PCT/US2020/050305

§ 371 Date: Feb. 17, 2022

Related U.S. Application Data
(60) Provisional application No. 62/899,297, filed on Sep. 12, 2019.

(51) Int. Cl. A61K 31/765; C12N 5/0784; A61K 31/4709; A61K 39/00; A61K 31/501; A61K 38/44; A61P 37/06

(52) U.S. Cl. CPC A61K 31/765; C12N 5/064; A61K 31/4709; A61K 39/0008; A61K 31/501; A61K 38/44; A61P 37/06

(57) ABSTRACT

The present invention relates to metabolite-based polymers and polymeric particles that serve as therapeutic agents, compositions comprising the same, and methods of use thereof.

### CROSS REFERENCE TO RELATED APPLICATIONS

This application claims priority to U.S. Provisional Application No. 62/899,297, filed Sep. 12, 2019 which is hereby incorporated by reference herein in its entirety.

### BACKGROUND

There is a rich history of successful drug delivery carriers made of biodegradable biomaterials that can modulate immune responses. Examples of such carriers include polyesters (e.g. poly (lactic-co-glycolic) acid (PLGA)—used in applications ranging from cancer to autoimmunity), and bi-lipid layer carriers (e.g. liposomes). Notably, these biomaterials degrade into metabolic by-products, which are capable of modulating the function of immune cells. For example, the degradation product of the drug delivery carrier poly(lactic acid) is lactic acid (a by-product of glycolysis), which can directly suppresses immune cells such as dendritic cells, (DCs—specialized immune cells responsible for inducing adaptive immune responses), macrophages (phagocytes, responsible for removing debris) and T-cell lymphocytes (responsible for mounting immune responses against foreign materials). Interestingly, there are several metabolites that are known to modulate function of immune cells including, succinate—activates DCs and lead to adaptive immune response, citrate—induces pro-inflammatory cytokines and reactive oxygen species, α-ketoglutarate—induces alternate activation (immunosuppressive phenotype) in macrophages through metabolic reprogramming, and polyunsaturated fatty acids (e.g. arachidonic acid C20:4(n-6))—blocks activation of DCs.

Antigen-specific immune suppression or tolerance is a highly attractive strategy to prevent inflammation-induced tissue damage in Rheumatoid Arthritis (RA). Although, there is insufficient knowledge about autoantigens in human RA, generating and expanding synovium-specific regulatory T-cells, and suppressing synovium-specific follicular T-helper cells, can provide global tissue-specific immunosuppression.

There is a great need to modulate the metabolism of immune cells, which controls their function including inflammation, suppression and tolerance. Further, there is a need in the art for compositions and methods that can generate tissue-specific tolerance by targeting specific pathways of immune cells, while providing multiple tissue antigens, without modulating systemic metabolism. The present invention satisfies this need.

### SUMMARY OF THE INVENTION

In one embodiment, the invention relates to a composition comprising an α-ketoglutarate polymer. In one embodiment, the polymer is a polymeric particle.

In one embodiment, the invention relates to a composition comprising an α-ketoglutarate polymer and at least one additional therapeutic agent for the treatment of a disease or disorder. In one embodiment, the at least one therapeutic agent is a peptide, a protein, an antibody, a nucleic acid molecule, a small molecule chemical compound, or any combination thereof. In one embodiment, the at least one therapeutic agent is PFK15, a connective tissue antigen, bc2, Cb-839, TRP2 or any combination thereof.

In one embodiment, the composition comprises an α-ketoglutarate polymeric particle comprising PFK15. In one embodiment, the composition comprises an α-ketoglutarate polymeric particle comprising bc2. In one embodiment, the composition comprises an α-ketoglutarate polymeric particle comprising a combination of PFK15 and bc2.

In one embodiment, the invention relates to a pharmaceutical composition comprising an α-ketoglutarate polymer. In one embodiment, the polymer is a polymeric particle. In one embodiment, the composition comprises an α-ketoglutarate polymeric particle comprising PFK15. In one embodiment, the composition comprises an α-ketoglutarate polymeric particle comprising bc2. In one embodiment, the composition comprises an α-ketoglutarate polymeric particle comprising a combination of PFK15 and bc2.

In one embodiment, the invention relates to a method of suppressing an immune response in a subject in need thereof, the method comprising administering a composition comprising a α-ketoglutarate polymer and at least one therapeutic agent for the treatment of a disease or disorder. In one embodiment, the at least one therapeutic agent is a peptide, a protein, an antibody, a nucleic acid molecule, a small molecule chemical compound, or any combination thereof. In one embodiment, the at least one therapeutic agent is PFK15, a connective tissue antigen, bc2, Cb-839, TRP2 or any combination thereof.

In one embodiment, the composition comprises an α-ketoglutarate polymeric particle comprising PFK15. In one embodiment, the composition comprises an α-ketoglutarate polymeric particle comprising bc2. In one embodiment, the composition comprises an α-ketoglutarate polymeric particle comprising a combination of PFK15 and bc2.

In one embodiment, the method comprises generating an immunosuppressive DC in a subject. In one embodiment, the DC comprises a phenotype of MHCII⁺CD86^{Lo}IL-10⁺IL-12p70^{Lo}. In one embodiment, the DC is MHCII⁺CD86^{Lo}IL-10⁺IL-12p70^{Lo}TNFα^{Lo}.

In one embodiment, the method comprises generating antigen-specific immunosuppressive T cell in the subject. In one embodiment, T cell is an antigen-specific regulatory T cell, antigen-specific T-helper 1, antigen-specific T-helper 17, antigen-specific T-helper 2 or antigen-specific T follicular helper cell.

In one embodiment, the invention relates to a method of treating a disease or disorder in a subject in need thereof, the method comprising administering a composition comprising a α-ketoglutarate polymer and at least one therapeutic agent for the treatment of a disease or disorder. In one embodiment, the at least one therapeutic agent is a peptide, a protein, an antibody, a nucleic acid molecule, a small molecule chemical compound, or any combination thereof. In one embodiment, the at least one therapeutic agent is PFK15, a connective tissue antigen, bc2, Cb-839, TRP2 or any combination thereof.

In one embodiment, the disease or disorder is an autoimmune disease. In one embodiment, the disease or disorder is rheumatoid arthritis (RA).

In one embodiment, the invention relates to a kit comprising an α-ketoglutarate polymer. In one embodiment, the polymer is a polymeric particle. In one embodiment, the kit comprises an α-ketoglutarate polymeric particle comprising PFK15. In one embodiment, the kit comprises an α-ketoglutarate polymeric particle comprising bc2. In one embodiment, the kit comprises an α-ketoglutarate polymeric particle comprising a combination of PFK15 and bc2.

### BRIEF DESCRIPTION OF THE DRAWINGS

The following detailed description of the invention will be better understood when read in conjunction with the appended drawings. For the purpose of illustrating the invention, several exemplary embodiments are shown in the drawings. It should be understood, however, that the invention is not limited to the precise arrangements and instrumentalities of the embodiments shown in the drawings.

FIG. 1A through FIG. 1E depict exemplary data demonstrating that polymeric particles of non-activating metabolites were synthesized. FIG. 1A depicts a schematic diagram demonstrating that a condensation reaction was used to generate poly-α-ketoglutarate (paKG) polymers. FIG. 1B depicts data demonstrating that 1H NMR confirmed the presence of 1,10 decanediol and α-ketoglutarate (αKG) in paKG, and show the release of αKG from particles. FIG. 1C depicts exemplary data demonstrating that microparticles are generated from paKG, as determined from electron microscopy and dynamic light scattering (FIG. 1D). FIG. 1E depicts data demonstrating that the paKG particles degrade over a period of time via hydrolysis of the ester bond as determined by weight loss experiments over 60 days, thereby releasing αKG (determined by 1H NMR data, upper line in FIG. 1i).

FIG. 2A through FIG. 2D depict experimental results demonstrating that paKG delivering PFK15 modulate adaptive immune responses in vitro. FIG. 2A depicts data demonstrating that paKG particles with rhodamine were phagocytosed by bone marrow derived DCs. FIG. 2B depicts data demonstrating that paKG particles by themselves do not activate bone marrow derived DCs, and in the presence of lipopolysaccharide (LPS) activate DCs. FIG. 2C depicts data demonstrating that paKG particles mediated intracellular delivery of PFK15 or PFK15+antigen BC2 even in the presence of LPS, significantly decrease activation of DCs as compared to PaKG+LPS as observed by CD86 expression. FIG. 2D depicts data demonstrating that paKG particles delivering PFK15 and BC2 intracellularly in DCs significantly increase Treg/Th1 ratios as compared to no treatment control in a syngeneic mixed lymphocyte reaction. All other conditions were not significantly different than no treatment control. (For all experiments, n>4, ±stderror, * p<0.05).

FIG. 3A through FIG. 3C depict experimental results demonstrating that paKG delivering PFK15 and bc2 modulate RA phenotype in DBA/1j CIA model. FIG. 3A depicts a diagram of the study design for vaccine prevention of RA in mice. FIG. 3B depicts normalized ankle thickness for different groups (n=5, average±stderror). FIG. 3C depicts exemplary images of RA phenotype in mice.

FIG. 4A through FIG. 4C depict experimental results demonstrating that paKG delivering PFK15 and bc2 modulate RA immune responses in vivo. FIG. 4A depicts anti-BC2 antibody titers. FIG. 4B depicts data demonstrating BC2-specific and proliferating Treg responses in the popliteal lymph nodes. FIG. 4C depicts BC2-specific proinflammatoryTh1 and Th17 responses in the inguinal lymph nodes in mice (n=5/group, average±stderror, *p<0.05).

FIG. 5A through FIG. 5E depict experimental results demonstrating that PFK15 blocks glycolysis in DCs in vitro. FIG. 5A depicts a schematic of PFK15 reprogramming glycolysis in DCs with PFKFB3 inhibition. FIG. 5B depicts exemplary flow cytometry data demonstrating that PFK15 is capable of decreasing CD86 expression in DCs. FIG. 5C and FIG. 5D depict the in vitro DC frequency. FIG. 5C depicts data demonstrating that PFK15 does not significantly impact CD86−MHCII+ DCs as compared to no treatment and LPS. FIG. 5D depicts data demonstrating that in presence of LPS, PFK15 significantly decreased CD86+MHCII+ DC populations as compared to LPS (n=5-11, avg±SEM, *, &—p<0.05; *—significantly different than no treatment; &—significantly different than LPS). FIG. 5E depicts data demonstrating that in the presence of LPS, PFK15 significantly lowered the glycolysis (ECAR) in DCs (n=30-36, avg±SEM, *, &, %—p<0.05; *—significantly different than no treatment, &—significantly different LPS, % significantly different than PFK15).

FIG. 6A through FIG. 6B depict experimental results demonstrating the characterization of paKG microparticles (MPs). FIG. 6A depicts the SEM of paKG, paKG(PFK15), and paKG(PFK15+bc2) MPs demonstrate spherical and heterogenous MPs (scale bar=10 μm, magnification 2000×). FIG. 6B depicts that DLS size distribution of paKG MPs determined the average of paKG(PFK15+bc2) to be 1.9±0.5.

FIG. 7A through FIG. 7F depict experimental results demonstrating that paKG(PFK15) MPs modulate DC metabolism in vitro. FIG. 7A depicts a schematic of paKG(PFK15) MPs modulating DC metabolism. FIG. 7B depicts data demonstrating that bone-marrow derived dendritic cells phagocytose paKG MPs (same field for TOP and BOTTOM image, paKG MP—red, nucleus—blue; gray=DIC filter, scale bar=10 mm), crossbar (yellow lines meet) in the bottom image shows same particle scene in different planes (x,y,z). FIG. 7C depicts data demonstrating that, as observed by metabolomics, paKG MPs modulate intracellular metabolite levels as compared to no treatment (out of 299 analyzed using LC-MS/MS), and their respective signaling pathways. paKG(PFK15) vs. no treatment—1—Citrate cycle, 2—Alanine, asparate and glutamate metabolism, 3—Glycolysis/Gluconegenesis, 4—Tryptophan, 5—Ascorbate and alderate metabolism, 6—Taurine and hypotaurine metabolism. paKG(PFK15) vs paKG—1—Histidine metabolism, 2—Arginine and proline metabolism, 3—Lysine degradation, 4—Alanine, asparate and glutamate metabolism, 5—Glutathione metabolism. Pathway impact—number of metabolites modified significantly in a pathway; log(p)—level of modulation. FIG. 7C depicts data demonstrating that in the presence of LPS, PFK15 significantly lowered the glycolysis (ECAR) in DCs (n=15-18, avg±SEM, *, &—p<0.05; *—significantly different than no treatment, &—significantly different than LPS.) FIG. 7E depicts a volcano plot of RNA-seq results display the fold changes (log 2 fold change) and the FDR-adjusted p values (−log 10 (FDR)) in genes that were upregulated (red) and downregulated (blue) when comparing paKG(PFK15) MP treated BMDCs to no treatment. FIG. 7F depicts a gene enrichment map is represented by the statistical significance (FDR-adjusted p value) of enriched pathways. The darker blue colors represent pathways that had an increased enrichment.

FIG. 8A through FIG. 8N depict experimental results demonstrating that paKG(PFK15+bc2) MPs alter adaptive immune responses in vitro. FIG. 8A depicts a schematic of paKG(PFK15+bc2) MPs reprogramming DC surface proteins for the modulation of adaptive immune responses. FIG. 8B depicts data demonstrating that DCs in the presence of paKG+LPS have less CD86 expression than LPS alone. FIG. 8C depicts data demonstrating that DCs in the presence of paKG(PFK15)+LPS and paKG(PFK15+bc2)+LPS have less CD86 expression than LPS alone. FIG. 8D depicts data demonstrating that in the presence of LPS, paKG MPs have less CD86 expression than LPS (n=5-11, avg±SEM, p<0.05; *=significantly different than LPS). FIG. 8E depicts data demonstrating that paKG MP groups have more CD86−MHCII+ DCs than CD86+MHCII+ DC populations. Y-axis to the left is for CD86−MHCII+ DCs and y-axis to the right is for CD86+MHCII+ DCs (n=6-12, avg±SEM, p<0.05; *=significantly different than CD86+MHCII+). (f) paKG(PFK15+bc2) MPs increase CD86−MHCII+ populations by 23-fold as compared to CD86+MHCII+ DC populations (n=5-11, avg±SEM, *, &, $—p<0.05; *=significantly different than LPS). FIG. 8G and FIG. 8H depict data demonstrating that CD86−MHCII+ DCs vs CD86+MHCII+ cytokine frequency (FIG. 8G), IL-10 (FIG. 8H) TNFα(n=3-6, avg±SEM, *—p<0.05; *=significantly different than CD86+MHCII+). FIG. 8I to FIG. 8N depict data demonstrating the in vitro T cell frequency in an allogenic MLR (FIG. 8I), Th2 cell frequency (FIG. 8J), CD4+ Treg cell frequency (FIG. 8K), proliferating CD4+ Treg cell frequency (FIG. 8L), Th1 cell frequency (FIG. 8M), Th17 cell frequency (FIG. 8N) proliferating Tc17 cell frequency (n=6, avg±SEM, *, &, $—p<0.05; *=significantly different than no treatment; &—significantly different than paKG-PFK15; $—significantly different than paKG).

FIG. 9A through FIG. 9G depict experimental results demonstrating that paKG(PFK15+bc2) MPs reverse inflammation in CIA mouse model. FIG. 9A depicts schema of in vivo CIA studies. FIG. 9B depicts data demonstrating the in vivo DC frequencies of CD86+, MHCII+, and CD11c+ in inguinal lymph nodes (n=9-14, avg±SEM, *—p<0.05; *=significantly different no treatment). FIG. 9C depicts data demonstrating the in vivo DC and macrophage frequency in injection site (n=4-8, avg±SEM, *—p<0.05; *=significantly different than paKG). FIG. 9D depicts data demonstrating the normalized percent change in back paw thickness after first dose of paKG(PFK15+bc2) (n=11-18, avg±SEM, *—p<0.05; *=significantly different than no treatment). FIG. 9E depicts data demonstrating the arthritic back paw scores. Arrows signify treatment days on day 35 and day 40 (n=7-9, avg±SEM *—p<0.05; *=significantly different than no treatment). FIG. 9F depicts data demonstrating the normalized percent change in weight as compared to baseline measurements (n=4-11, avg±SEM, p<0.05—*=significantly different than paKG(PFK15)). FIG. 9G depicts representative front paw images of no treatment, paKG, paKG(PFK15), and paKG(PFK15+bc2).

FIG. 10A through FIG. 10C depict experimental results demonstrating that paKG(PFK15+bc2) alter adaptive immune responses in vivo. FIG. 10A depicts data demonstrating in vivo T cell frequencies in the popliteal lymph nodes (n=3-4; avg±SEM, p<0.05—*=significantly different than no treatment). FIG. 10B depicts data demonstrating ex vivo T cell frequencies in the cervical lymph nodes (n=18-22; avg±SEM, p<0.05—*=significantly different than no treatment). FIG. 10C depicts a heatmap of inflammatory and anti-inflammatory CD4+ and CD8+ cell types in ex vivo T cell study.

FIG. 11A through FIG. 11D depict experimental results demonstrating that paKG(PFK15+bc2) MPs modulate cytokine and adaptive T cell responses. FIG. 11A depicts data demonstrating IL-12 frequency in CD86−MHCII+ DCs vs CD86+MHCII+ (n=3-6, avg±SEM). FIG. 11B to FIG. 11D depict data demonstrating in vitro T cell frequencies (FIG. 11B), activated Th2 cell types (FIG. 11C), activated and proliferating Foxp3+RorγT+ cell frequency—Tregs that suppress Th17 cell types (FIG. 11D), CD8+ Treg cell frequency (n=6, avg±SEM, *, $—p<0.05; *=significantly different than treatment; $—significantly different than paKG).

FIG. 12A and FIG. 12B depict experimental results demonstrating that paKG(PFK15+bc2) modulate RA immune responses in in vivo prevention studies. FIG. 12A depicts data demonstrating bc2-specific and proliferating Treg responses in the popliteal lymph nodes. FIG. 12B depicts data demonstrating bc2-specific pro-inflammatory Th1 and Th17 responses in the inguinal lymph nodes (n=5, average±stderror, * p<0.05).

FIG. 13A and FIG. 13B depict experimental results demonstrating that paKG(PFK15) and paKG MPs modulate inflammation in CIA mouse model. FIG. 13A depicts data demonstrating the normalized percent change in back paw thickness after first dose (n=8-18; avg±SEM). FIG. 13B depicts data demonstrating the arthritic back paw scores. Arrows signify treatment days on day 35 and day 40 (n=4-7, avg±SEM).

FIG. 14A through FIG. 14D depict experimental results demonstrating that paKG(PFK15) and paKG MPs modulate inflammation in CIA mouse model. FIG. 14A depicts data demonstrating the in vivo T cell frequencies in the spleen (n=3-4; avg±SEM, p<0.05—*=significantly different than no treatment). FIG. 14B through FIG. 14D depict data demonstrating CD8+ T cell tSNE plots, (FIG. 14B) CD25+ and Ki67+, (FIG. 14C) CD44+ and CD62L+ (FIG. 14D) PD-1+ (n=4).

### DETAILED DESCRIPTION

The present invention relates to compositions comprising a metabolite-based polymer and methods for their use as therapeutic molecules for altering an immune response in a subject in need thereof.

In one embodiment, the composition comprising a metabolite-based polymer comprises at least one agent. In one embodiment, the composition comprises at least one therapeutic agent for the treatment of a disease or disorder. In one embodiment, the composition comprises at least one antigen. In one embodiment, the composition comprises at least one of 1-(4-pyridinyl)-3-(2-quinolinyl)-2-propen-1-one (PFK15), a connective tissue antigen, bovine collagen type II (bc2) antigen, Cb-839, and TRP2.

In one embodiment, the metabolite-based polymer is a polymeric particle. In one embodiment, the metabolite-based polymeric particles encapsulate at least one agent. In one embodiment, the metabolite-based polymeric particles encapsulate at least one therapeutic agent for the treatment of a disease or disorder. In one embodiment, the metabolite-based polymeric particles encapsulate at least one antigen. In one embodiment, the metabolite-based polymeric particles encapsulate at least one of PFK15, a connective tissue antigen, bc2, Cb-839, and TRP2.

In one embodiment, the invention relates to methods of using the metabolite-based polymeric compositions of the present invention to release at least one agent. In various embodiments, at least one agent is released intracellularly in cells or extracellularly. In one embodiment, at least one agent is released in a sustained manner.

In one embodiment, the invention relates to methods of using the metabolite-based polymeric compositions to modulate a biological pathway or process. In various embodiments, the pathway or process is glycolysis or the glutaminase pathway.

In one embodiment, the invention relates to methods of using the metabolite-based polymeric compositions to induce immune suppression. In one embodiment, immune suppression is induced in immune cells, including but not limited to, dendritic cells and T cells.

In on embodiment, the invention provides a method of generating of a dendritic cell having an immunosuppressive DC phenotype in a subject in need thereof. In one embodiment, the immunosuppressive DC phenotype is MHCII⁺CD86^{Lo}IL-10⁺IL-12p70^{Lo}. In one embodiment, the DC is TNFα^{Lo}.

In on embodiment, the invention provides a method of generating of a T cell having an immunosuppressive phenotype in a subject in need thereof. In one embodiment, the immunosuppressive T cell is a regulatory T cell (Treg). In one embodiment, the immunosuppressive Treg is specific for a disease or disorder.

The present invention also provides a method of treating or preventing a disease or disorder in a subject. In one embodiment, the method comprises administering an effective amount of a composition comprising a metabolite-based polymer described herein to a subject. In one embodiment, the composition comprises a therapeutic agent for the treatment of the patient's disease or disorder. In one embodiment, the disease or disorder is an inflammatory or autoimmune disease or disorder. In one embodiment, the disease or disorder is rheumatoid arthritis (RA).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, +5%, ±1%, or +0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “analog” as used herein generally refers to compounds that are generally structurally similar to the compound of which they are an analog, or “parent” compound. Generally, analogs will retain some characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while at least one distinct biological activities of the parent are unaffected in the “analog.”

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes, examination of cells, cytogenetics, and immunophenotyping of blood cells.

As used herein, “conjugated” refers to covalent attachment of one molecule to a second molecule.

The term “comparator” describes a material comprising none, or a normal, low, or high level of one of more of the marker (or biomarker) expression products of at least one the markers (or biomarkers) of the invention, such that the comparator may serve as a control or reference standard against which a sample can be compared.

As used herein, the term “derivative” includes a chemical modification of a polypeptide, polynucleotide, or other molecule. In the context of this invention, a “derivative polypeptide,” for example, one modified by glycosylation, pegylation, or any similar process, retains binding activity. For example, the term “derivative” of binding domain includes binding domain fusion proteins, variants, or fragments that have been chemically modified, as, for example, by addition of at least one polyethylene glycol molecules, sugars, phosphates, and/or other such molecules, where the molecule or molecules are not naturally attached to wild-type binding domain fusion proteins. A “derivative” of a polypeptide further includes those polypeptides that are “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

As used herein, the term “diagnosis” means detecting a disease or disorder or determining the stage or degree of a disease or disorder. Usually, a diagnosis of a disease or disorder is based on the evaluation of at least one factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein “endogenous” refers to any material from or produced inside an organism, cell, tissue or system.

As used herein “exogenous” refers to any material from or produced outside an organism, cell, tissue or system.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means. “Polynucleotide” includes cDNA, RNA, DNA/RNA hybrid, anti-sense RNA, siRNA, miRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of at least one nucleotides, or fusion to other polynucleotide sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “pharmaceutically acceptable carrier” means a pharmaceutically acceptable material, composition or carrier, such as a liquid or solid filler, diluent, excipient, solvent or encapsulating material, involved in carrying or transporting a metabolite-based polymer or polymeric particle of the present invention within or to the subject such that it can perform its intended function. Typically, such metabolite-based polymers or polymeric particles are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the metabolite-based polymer or polymeric particle, and are physiologically acceptable to the subject. Supplementary active compounds can also be incorporated into the compositions.

The terms “subject,” “patient,” “individual,” and the like are used interchangeably herein, and refer to any animal, or cells thereof whether in vitro or in situ, amenable to the methods described herein. Thus, the individual may include, for example, dogs, cats, pigs, cows, sheep, goats, horses, rats, monkeys, and mice and humans. In some non-limiting embodiments, the patient, subject or individual is a human.

A “therapeutic” treatment is a treatment administered to a subject who exhibits a sign or symptom of a disease or disorder, for the purpose of diminishing or eliminating the sign or symptom of the disease or disorder.

The terms “treat,” “treating,” and “treatment,” refer to therapeutic or preventative measures described herein. The methods of “treatment” employ administration to a subject, in need of such treatment, a composition of the present invention. For example, a subject afflicted with a disease or disorder, or a subject who ultimately may acquire such a disease or disorder, in order to prevent, cure, delay, reduce the severity of, or ameliorate at least one sign or symptom of the disease or disorder.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “aromatic” refers to a carbocycle or heterocycle with at least one polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing at least one rings (typically one, two or three rings) wherein such rings may be attached together in a pendent manner, such as a biphenyl, or may be fused, such as naphthalene. Examples include phenyl, anthracyl, and naphthyl.

As used herein, the term “aryl-(C₁-C₃)alkyl” means a functional group wherein a one to three carbon alkylene chain is attached to an aryl group, e.g., —CH₂CH₂-phenyl. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include at least one rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on experiments demonstrating that 1-(4-pyridinyl)-3-(2-quinolinyl)-2-propen-1-one (PFK15) and bovine collagen type II (bc2) antigen, when delivered together in a particle format, lead to the generation of adaptive responses in vivo.

The present invention is based, in part, on the development of novel active metabolite-based polymers and polymeric particles and their use as carriers for the delivery of therapeutic agents. In one embodiment, metabolite-based poly-α-ketoglutarate (paKG) polymeric particles encapsulating PFK15 and bc2 antigen are capable of generating tissue-specific tolerance by targeting specific pathways of immune cells, while providing multiple tissue antigens, without modulating systemic metabolism.

The invention, in part, provides a method for generating immunosuppressive dendritic cells (DCs), in vivo, which are capable of delivering both antigens and immunosuppressive agents.

### Metabolite-Based Polymers

In one embodiment, the invention provides active metabolite-based polymers and polymeric particles that can serve as carriers for at least one therapeutic agent. In one embodiment, the metabolite is α-ketoglutarate, and the metabolite-based polymer is a polymer of α-ketoglutarate (paKG).

In one embodiment, the metabolite-based polymer forms a polymeric particle. In one embodiment, the polymeric particle provided herein is a nanoparticle having any suitable size. In one embodiment, the nanoparticle provided herein has an average diameter of about 5 nm to about 1000 nm. In one embodiment, the nanoparticle has an average diameter of about 5 nm to about 500 nm. In one embodiment, the nanoparticle has an average diameter of about 10 nm to about 200 nm. In one embodiment, the nanoparticle has an average diameter of about 20 nm to about 200 nm. In one embodiment, the nanoparticle has an average diameter of about 10 nm to about 100 nm. In one embodiment, the nanoparticle has an average diameter of about 20 nm to about 100 nm.

In one embodiment, the polymeric particle provided herein is a microparticle having any suitable size. In one embodiment, the microparticle provided herein has an average diameter of about 1 μm to about 500 μm. In one embodiment, the nanoparticle has an average diameter of about 1 μm to about 200 μm. In one embodiment, the nanoparticle has an average diameter of about 1 μm to about 100 μm. In one embodiment, the nanoparticle has an average diameter of about 1 μm to about 50 μm. In one embodiment, the nanoparticle has an average diameter of about 1 μm to about 20 μm.

In one embodiment, the metabolite-based polymer or polymeric particle functions as a carrier for at least one therapeutic agent. In one embodiment, the at least one therapeutic agent is encapsulated in a polymeric particle of the invention. In one embodiment, the at least one therapeutic agent is conjugated to the surface of a metabolite-based polymer or polymeric particle of the invention. In one embodiment, the at least one therapeutic agent is administered in combination with a metabolite-based polymer or polymeric particle of the invention. The metabolite-based polymer or polymeric particle can function as carriers for hydrophobic or hydrophilic agents.

In one embodiment, the metabolite-based polymer or polymeric particle has at least one of high loading and high encapsulation efficiency of the at least one therapeutic agent. Encapsulation efficiency is the percentage of drug that is successfully entrapped into a polymeric particle. In one embodiment, encapsulation efficiency (% EE) is calculated by (total drug added—free non-entrapped drug) divided by the total drug added. Loading capacity is the amount of drug loaded per unit weight of a polymeric particle, indicating the percentage of mass of the polymeric particle that is due to the encapsulated drug. In one embodiment, loading capacity (% LC) can be calculated by the amount of total entrapped drug divided by the total polymeric particle weight. In one embodiment, the polymeric particle has about 50% to about 100% encapsulation efficiency of the at least one therapeutic agent. In one embodiment, the polymeric particle has about 60% to about 100% encapsulation efficiency of the at least one therapeutic agent.

In one embodiment, the composition comprising the metabolite-based polymer or polymeric particle of the invention comprises at least one agent. For example, the metabolite-based polymer or polymeric particle may be conjugated to, loaded with, or encapsulate, at least 1, 2, 3, 4, 5, 6, 7, 8, 9, 10, 11, 12, 13, 14, 15, 16, 17, 18, 19, 20, 25, 30, 35, 40, 45, 50 or more than 50 different agents. In one embodiment, the metabolite-based polymer or polymeric particle may be administered in combination with at least 1, 2, 3, 4, 5, 6, 7, 8, 9, 10, 11, 12, 13, 14, 15, 16, 17, 18, 19, 20, 25, 30, 35, 40, 45, 50 or more than 50 different agents.

In one embodiment, the at least one agent is a therapeutic agent. Therefore, in one embodiment, the at least one agent is useful for the treatment or prevention of a disease or disorder. Diseases and disorders that can be treated using the metabolite-based polymer or polymeric particles of the invention include, but are not limited to, inflammatory or autoimmune diseases and disorders. In one embodiment, the composition comprising the metabolite-based polymer or polymeric particle of the invention comprises at least one of 1-(4-pyridinyl)-3-(2-quinolinyl)-2-propen-1-one (PFK15), CB-839, a glutaminase inhibitor, a glycolytic inhibitor, or retinoic acid.

In one embodiment, the at least one agent is an antigen. Exemplary antigens that can be conjugated to or encapsulated by the metabolite-based polymer or polymeric particle of the invention include, but are not limited to, self-antigens, as described elsewhere herein. In one embodiment, the antigen is useful for breaking tolerance. In one embodiment, the composition comprising the metabolite-based polymer or polymeric particle of the invention comprises at least one of a connective tissue antigen including, but not limited to, collagen type I, II, III and IV antigens, albumin, hsp60 peptides, or human cartilage glycoprotein-39.

In one embodiment, the composition comprising the metabolite-based polymer or polymeric particle of the invention comprises at least one antigen and at least one therapeutic agent. For example, in one embodiment, the composition comprising the metabolite-based polymer or polymeric particle of the invention comprises at least one of PFK15, CB-839, a glutaminase inhibitor, a glycolytic inhibitor, or retinoic acid and at least one of a connective tissue antigen including, but not limited to, collagen type I, II, III or IV antigen, albumin, hsp60 peptides, human cartilage glycoprotein-39. In one embodiment, the composition comprising the metabolite-based polymer or polymeric particle of the invention comprises bovine collagen type 2 (bc2) antigen and PFK15.

### Proteins and Peptides

In one embodiment, the composition comprising the metabolite-based polymer or polymeric particle of the invention comprises a protein or peptide. The peptide of the present invention may be made using chemical methods. For example, peptides can be synthesized by solid phase techniques cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

In one embodiment, the peptide is made by recombinant means or by cleavage from a longer polypeptide. The composition of a peptide may be confirmed by amino acid analysis or sequencing.

The peptides can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides may include unnatural amino acids formed by post-translational modification or by introducing unnatural amino acids during translation. A variety of approaches are available for introducing unnatural amino acids during protein translation.

A peptide or protein of the invention may be modified, e.g., phosphorylated, using conventional methods.

In one embodiment, the composition comprising the metabolite-based polymer or polymeric particle of the invention comprises a cyclic peptide. Cyclization of peptide may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptide may be synthesized by conventional techniques. For example, the peptides or chimeric proteins may be synthesized by chemical synthesis using solid phase peptide synthesis or solution phase synthesis methods. By way of example, a peptide may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain a peptide fused to the selected protein or marker protein as described herein. Examples of proteins which may be used to prepare fusion proteins include immunoglobulins, glutathione-S-transferase (GST), hemagglutinin (HA), and truncated myc.

Peptides may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. Libraries may also be constructed by concurrent synthesis of overlapping peptides.

Peptides and proteins may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one embodiment, the composition comprising the metabolite-based polymer or polymeric particle of the invention comprises an antibody, or antibody fragment, that specifically binds to a target. In some embodiments, the antibody can inhibit the target to provide a beneficial effect.

### Antibody Therapeutic Agents

The invention also includes a metabolite-based polymer or polymeric particle comprising an antibody, or antibody fragment, that specifically binds to a target. In some embodiments, the antibody can inhibit the target to provide a beneficial effect.

In various embodiments, the antibody is a monoclonal antibody, polyclonal antibody, immunologically active fragments of an antibody (e.g., a Fab or (Fab)₂ fragment), antibody heavy chain, antibody light chain, humanized antibody, genetically engineered single chain FV molecule, or a chimeric antibody. In some embodiments, the chimeric antibody contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using an intact polypeptide, or a fragment of the polypeptide containing an immunizing antigen of interest. The polypeptide or fragment used to immunize an animal may be obtained from the translation of RNA, or can be synthesized chemically, and can be conjugated to a carrier protein, if desired. Suitable carriers that may be chemically coupled to polypeptides and fragment thereof include bovine serum albumin, thyroglobulin, and keyhole limpet hemocyanin. The coupled polypeptide can then be used to immunize an animal (e.g., a mouse, a rat, or a rabbit).

### Antigens

In one embodiment, the composition comprising the metabolite-based polymer or polymeric particle of the invention comprises a desirable antigen of interest, including, but not limited to a self-antigen. Exemplary self-antigens include, but are not limited to, a connective tissue antigen including, but not limited to, collagen type I, II, III and IV antigens, albumin, hsp60 peptides, and human cartilage glycoprotein-39.

### Small Molecule Therapeutic Agents

In various embodiments, the therapeutic agent is a small molecule chemical compound. When the therapeutic agent is a small molecule, the small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule therapeutic agent comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determine the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the agents depicted here, as well as the non-salt and non-solvate form of the agents, as is well understood by the skilled artisan. In some embodiments, the salts of the agents of the invention are pharmaceutically acceptable salts.

Where tautomeric forms may be present for any of the agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the agents described. The recitation of the structure or name herein is intended to embrace all possible stereoisomers of agents depicted. All forms of the agents are also embraced by the invention, such as crystalline or non-crystalline forms of the agents. Compositions comprising an agent of the invention are also intended, such as a composition of substantially pure agent, including a specific stereochemical form thereof, or a composition comprising mixtures of agents of the invention in any ratio, including two or more stereochemical forms, such as in a racemic or non-racemic mixture.

In one embodiment, the composition comprising the metabolite-based polymer or polymeric particle of the invention comprises an analog or derivative of a therapeutic agent described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by at least one chemical reactions. As such, an analog can be a structure having a structure similar to that of the small molecule therapeutic agents described herein or can be based on a scaffold of a small molecule therapeutic agent described herein, but differing from it in respect to certain components or structural makeup, which may have a similar or opposite action metabolically. An analog or derivative of any of a small molecule agent in accordance with the present invention can be used to treat a disease or disorder.

In one embodiment, the small molecule therapeutic agents described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having at least one hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In some embodiments, the therapeutic agent is an isolated nucleic acid. In various embodiments, the isolated nucleic acid molecule is a DNA molecule or an RNA molecule. In various embodiments, the isolated nucleic acid molecule is a cDNA, mRNA, miRNA, siRNA, antagomir, antisense molecule, or CRISPR guide RNA molecule. In one embodiment, the isolated nucleic acid molecule encodes a therapeutic peptide. In some embodiments, the therapeutic agent is an siRNA, miRNA, sgRNA or antisense molecule, which inhibits a targeted nucleic acid. In one embodiment, the nucleic acid comprises a promoter/regulatory sequence such that the nucleic acid is capable of directing expression of the nucleic acid. Thus, in one embodiment, the composition comprising the metabolite-based polymer or polymeric particle of the invention comprises an expression vector, and the invention comprises a method for the introduction of exogenous DNA into cells with concomitant expression of the exogenous DNA in the cells.

In one embodiment of the invention, a targeted gene or protein, can be inhibited by way of inactivating and/or sequestering the targeted gene or protein. As such, inhibiting the activity of the targeted gene or protein can be accomplished by using an antisense nucleic acid molecule or a nucleic acid molecule encoding a transdominant negative mutant.

In one embodiment, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. In one embodiment, an siRNA comprises a chemical modification that aids in intravenous systemic delivery. Optimizing siRNAs involves consideration of overall G/C content, C/T content at the termini, Tm and the nucleotide content of the 3′ overhang. Therefore, the present invention also includes methods of decreasing levels of the expression products (i.e., mRNA and protein) of the target gene of interest using the metabolite-based polymers or polymeric particles of the invention for delivery of RNAi technology.

In one embodiment, the composition comprising the metabolite-based polymer or polymeric particle of the invention comprises a vector comprising an siRNA or antisense polynucleotide. In one embodiment, the siRNA or antisense polynucleotide is capable of inhibiting the expression of a target polypeptide. The incorporation of a desired polynucleotide into a vector and the choice of vectors is well-known in the art.

In certain embodiments, the expression vectors described herein encode a short hairpin RNA (shRNA) therapeutic agent. shRNA molecules are well known in the art and are directed against the mRNA of a target, thereby decreasing levels of the expression products (i.e., mRNA and protein) of the target gene of interest. In certain embodiments, the encoded shRNA is expressed by a cell, and is then processed into siRNA. For example, in certain instances, the cell possesses native enzymes (e.g., dicer) that cleaves the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells contacted with a composition comprising the metabolite-based polymer or polymeric particle of the invention. In one embodiment, the selectable marker may be carried on a separate piece of DNA and also be contained within a polymeric particle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one embodiment, a polymeric particle of the invention may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art. In a particular embodiment, the vector is a vector useful for transforming animal cells.

In one embodiment, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, and other techniques known in the art. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art. Antisense nucleic acids are DNA or RNA molecules that are complementary to at least a portion of a specific mRNA molecule. In the cell, antisense nucleic acids hybridize to the corresponding mRNA, forming a double-stranded molecule thereby inhibiting the translation of genes.

The use of antisense methods to inhibit the translation of genes is known in the art. Such antisense molecules may be provided to the cell via genetic expression using DNA encoding the antisense molecule.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides.

In one embodiment of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents or they may be genetically expressed from DNA encoding them.

In one embodiment, the therapeutic agent may comprise at least one component of a CRISPR-Cas system, where a guide RNA (gRNA) targeted to a gene encoding a target molecule, and a CRISPR-associated (Cas) peptide form a complex to induce mutations within the targeted gene. In one embodiment, the therapeutic agent comprises a gRNA or a nucleic acid molecule encoding a gRNA. In one embodiment, the therapeutic agent comprises a Cas peptide or a nucleic acid molecule encoding a Cas peptide.

### Pharmaceutical Compositions

The present invention provides pharmaceutical compositions comprising a metabolite-based polymer or polymeric particle of the invention. The relative amounts of the metabolite-based polymer or polymeric particle, any pharmaceutically acceptable carrier, and any additional ingredients in a pharmaceutical composition of the invention will vary, depending upon the identity, size, and condition of the subject treated and further depending upon the route by which the composition is to be administered.

The formulations of the pharmaceutical compositions described herein may be prepared by any method known or hereafter developed in the art of pharmacology. In general, such preparatory methods include the step of bringing the active ingredient into association with a carrier or at least one other accessory ingredients. Said compositions may comprise additional medicinal agents, pharmaceutical agents, carriers, buffers, adjuvants, dispersing agents, diluents, and the like depending on the intended use and application.

Examples of suitable pharmaceutical carriers, excipients and/or diluents are well known in the art and include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Pharmaceutically acceptable carriers for liquid formulations are aqueous or non-aqueous solutions, suspensions, emulsions or oils, Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, turmeric oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the biologically active compound of the invention, retains the biological activity. Preparations for parenteral administration may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like, in addition, the pharmaceutical composition of the present invention might comprise proteinaceous carriers, e.g., serum albumin or immunoglobulin, preferably of human origin.

A metabolite-based polymer or polymeric particle may be administered alone, or in combination with other drugs and/or agents as pharmaceutical compositions. The composition may contain at least one added materials such as carriers and/or excipients. As used herein, “carriers” and “excipients” generally refer to substantially inert, non-toxic materials that do not deleteriously interact with other components of the composition. These materials may be used to increase the amount of solids in particulate pharmaceutical compositions, such as to form a powder of drug particles. Examples of suitable carriers include water, silicone, gelatin, waxes, and the like.

Examples of normally employed “excipients,” include pharmaceutical grades of mannitol, sorbitol, inositol, dextrose, sucrose, lactose, trehalose, dextran, starch, cellulose, sodium or calcium phosphates, calcium sulfate, citric acid, tartaric acid, glycine, high molecular weight polyethylene glycols (PEG), and the like and combinations thereof. In one embodiment, the excipient may also include a charged lipid and/or detergent in the pharmaceutical compositions. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN surfactants (Atlas Chemical Industries, Wilmington, Del.), polyoxyethylene ethers, for example, Brij®, pharmaceutically acceptable fatty acid esters, for example, lauryl sulfate and salts thereof (SDS), and the like. Such materials may be used as stabilizers and/or anti-oxidants. Additionally, they may be used to reduce local irritation at the site of administration.

In at least one embodiment, the composition is formulated in a lyophilized form. In certain embodiments, the lyophilized formulation of the composition allows for maintaining structure and achieving remarkably superior long-term stability conditions which might occur during storage or transportation of the composition.

### Modified Cells

In one embodiment, the invention relates to a cell that has been modified using a metabolite-based polymer or polymeric particle of the invention. Non-limiting examples of cell types that can be modified according to the methods of the invention include, but are not limited to, epithelial cells, myeloid cells, basophils, neutrophils, eosinophils, monocytes, macrophages, natural killer (NK) cells, macrophages, dendritic cells (DC), lymphocytes, innate lymphoid cells (ILC), B cells, and T cells. Measurable parameters that may be altered in response to the exposure of cells to a polymeric particle of the invention include, but are not limited to, epithelial permeability, tight junction complex composition and expression, mucus production (e.g., expression of mucins, etc.), expression of inflammatory cytokines and chemokines (e.g., IL-1, IL-6, IL-12, IL-13, IL-17, IL-22, IL-23, IL-25, IL-33, TSLP, TNF, IFNg, etc.), expression of anti-inflammatory cytokines and chemokines (e.g., IL-10, TGF-b, etc.), stimulation of TLRs and other receptors (e.g., dectins, NODs, RLRs, etc.), maturation (e.g., expression of MHC-II, CD80/86, CD40, etc.), differentiation, ability to activate T cells, ability to differentiate T cells into effector subsets (TH1, TH2, TH9, TH17, Th22, Tregs, etc.), ability of innate lymphoid cells (ILC) to produce cytokines (e.g., IL-22, IL-17, IL-13, IFNg, etc.) when stimulated by IL-23, TSLP, IL-33, IL-25, etc., ability of B cells to produce antibodies of different isotypes (e.g., IgA, IgM, IgG, IgE, IgD, etc.), ability of T cells to become activated and differentiated into different effector lineages (TH1, TH2, TH9, TH17, Th22, Tregs, etc.) with, or without, stimulated by aCD3, aCD28, DCs, etc., or combinations thereof.

In on embodiment, the invention provides a method of generating of a dendritic cell having an immunosuppressive DC phenotype in a subject in need thereof. In one embodiment, the immunosuppressive DC phenotype is MHCII⁺CD86^{Lo}IL-10⁺IL-12p70^{Lo}. In one embodiment, the DC is TNFα^{Lo}.

In on embodiment, the invention provides a method of generating of a T cell having an immunosuppressive phenotype in a subject in need thereof. In one embodiment, the immunosuppressive T cell is a regulatory T cell (Treg), antigen-specific T-helper 1, antigen-specific T-helper 17, antigen-specific T-helper 2 or antigen-specific T follicular helper cell. In one embodiment, the immunosuppressive T cell is specific for a disease or disorder.

### Methods of Making Metabolic Polymers

In one embodiment, the invention provides a method for making a metabolite-based polymer or polymeric particle of the invention, having a carboxylic group. In one embodiment, the method comprises forming a polymer of the metabolite. In one embodiment, the method comprises forming a particle of the polymer. In one embodiment, forming a polymer of the metabolite comprises mixing the metabolite comprising a carboxylic group and 1,12 dodecanediol, and heating the mixture. In one embodiment, forming a particle of the polymer comprises a water-oil emulsion. In one embodiment, forming a particle of the polymer comprises a water-oil-water emulsion.

In one embodiment, the polymeric particle is formed in a solution comprising at least one agent. For example, the polymeric particle may be generated in the presence of at least 1, 2, 3, 4, 5, 6, 7, 8, 9, 10, 11, 12, 13, 14, 15, 16, 17, 18, 19, 20, 25, 30, 35, 40, 45, 50 or more than 50 different agents to generate a loaded polymeric particle.

### Methods of Use

The invention relates to methods of using a composition comprising the metabolite-based polymer or polymeric particle of the invention. In some embodiments, the composition is a pharmaceutical composition. In some embodiments, the method includes that administration of a composition comprising a metabolite-based polymer or polymeric particle comprising a therapeutic agent.

In one embodiment, the invention is a method of suppressing an immune response in a subject in need thereof. In one embodiment, the invention relates to methods of using the composition comprising the metabolite-based polymer or polymeric particle of the present invention to release metabolic inhibitors intracellularly in at least one immune cell. In one embodiment, the invention relates to methods of using the composition comprising the polymeric particle of the present invention to release metabolic inhibitors extracellularly.

In one embodiment, the invention relates to methods of using the composition comprising the polymeric particle, of the present invention to induce immune suppression in immune cells, including but not limited to, dendritic cells and T cells. In one embodiment, the invention provides a method of generating of a dendritic cell having an immunosuppressive DC phenotype in a subject in need thereof. In one embodiment, the immunosuppressive DC phenotype is MHCII⁺CD86^{Lo}IL-10⁺IL-12p70^{Lo}. In one embodiment, the DC is TNFα^{Lo}. In one embodiment, the invention provides a method of generating of a T cell having an immunosuppressive phenotype in a subject in need thereof. In one embodiment, the immunosuppressive T cell is a regulatory T cell (Treg). In one embodiment, the immunosuppressive Treg is specific for a disease or disorder.

The present invention also provides a method of treating or preventing a disease or disorder in a subject. In one embodiment, the method comprises administering an effective amount of a composition comprising the nanoparticle of the invention to a subject in need thereof. In one embodiment, the polymeric particle comprises at least one therapeutic agent to treat the patient's disease or disorder.

Exemplary diseases or disorders that can be treated using the compositions and methods of the invention include, but are not limited to, inflammatory diseases and disorders, and autoimmune diseases and disorders. Exemplary diseases that can be treated using the compositions and methods of the invention include, but are not limited to, rheumatoid arthritis/seronegative arthropathies, osteoarthritis, inflammatory bowel disease, systemic lupus erythematosis, iridoeyelitis/uveitistoptic neuritis, idiopathic pulmonary fibrosis, systemic vasculitis/Wegener's gramilornatosis, sarcoidosis, including, but not limited to, rheumatoid arthritis/seronegative arthropathies, osteoarthritis, inflammatory bowel disease, systemic lupus erythematosis, iridoeyelitis/uveitistoptic neuritis, idiopathic pulmonary fibrosis, systemic vasculitis/Wegener's gramilornatosis, sarcoidosis, myocarditis, postmyocardial infarction syndrome, postpericardiotomy syndrome, subacute bacterial endocarditis (SBE), anti-glomerular basement membrane nephritis, interstitial cystitis, lupus nephritis, autoimmune hepatitis, primary biliary cholangitis (PBC), primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus vulgaris, Pityriasis lichenoides et varioliformis acuta, Mucha-Habermann disease, psoriasis, systemic scleroderma, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, Coeliac disease, Crohn's disease, microscopic colitis, ulcerative colitis, antiphospholipid syndrome (APS, APLS), aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, Schnitzler syndrome, undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (Anti-NMDA) receptor encephalitis, balo concentric sclerosis, Bickerstaff s encephalitis, chronic inflammatory demyelinating polyneuropathy, Guillain-Barre syndrome, Hashimoto's encephalopathy, idiopathic inflammatory demyelinating diseases, Lambert-Eaton myasthenic syndrome, multiple sclerosis, pattern II, Oshtoran Syndrome, pediatric autoimmune neuropsychiatric disorder associated with Streptococcus (PANDAS), progressive inflammatory neuropathy, restless leg syndrome, stiff person syndrome, sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Meniere's disease, Behget's disease, eosinophilic granulomatosis with polyangiitis (EGPA), giant cell arteritis, granulomatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumatic, urticarial vasculitis, vasculitis, and primary immune deficiency. In one embodiment, the disease is rheumatoid arthritis.

In various embodiments, the composition comprising the metabolite-based polymer of the invention is administered to a subject in need in a wide variety of ways. In various embodiments, the polymeric particle, or pharmaceutical composition comprising the polymeric particle, of the invention is administered orally, intraoperatively, intravenously, intravascularly, intramuscularly, subcutaneously, intracerebrally, intraperitoneally, by soft tissue injection, by surgical placement, by arthroscopic placement, and by percutaneous insertion, e.g., direct injection, cannulation or catheterization. Any administration may be a single administration of a composition of invention or multiple administrations. Administrations may be to single site or to more than one site in the subject being treated. Multiple administrations may occur essentially at the same time or separated in time.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are preferably administered by i.v. injection.

The composition comprising the polymeric particle described herein can be incorporated into any formulation known in the art. For example, the polymeric particle may be incorporated into formulations suitable for oral, parenteral, intravenous, subcutaneous, percutaneous, topical, buccal, or another route of administration. Suitable compositions include, but are not limited to, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

In the method of treatment, the administration of the composition of the invention may be for either “prophylactic” or “therapeutic” purpose. When provided prophylactically, the composition of the present invention is provided in advance of any sign or symptom, although in particular embodiments the invention is provided following the onset of at least one sign or symptom to prevent further signs or symptoms from developing or to prevent present signs or symptoms from becoming more severe. The prophylactic administration of the composition serves to prevent or ameliorate subsequent signs or symptoms. When provided therapeutically, the pharmaceutical composition is provided at or after the onset of at least one sign or symptom. Thus, the present invention may be provided either prior to the anticipated exposure to a disease-causing agent or disease state or after the initiation of the disease or disorder.

### Kits of the Invention

The invention also includes a kit comprising compounds useful within the methods of the invention and an instructional material that describes, for instance, the method of administering the polymeric particles and compositions as described elsewhere herein. The kit may comprise formulations of a pharmaceutical composition comprising the active ingredient combined with a pharmaceutically acceptable carrier, such as sterile water or sterile isotonic saline. The kit may comprise injectable formulations that may be prepared, packaged, or sold in unit dosage form, such as in ampules or in multi dose containers containing a preservative. The kit may comprise formulations including, but not limited to, suspensions, solutions, emulsions in oily or aqueous vehicles, pastes, and implantable sustained-release or biodegradable formulations. Such formulations may further comprise at least one additional ingredient including, but not limited to, suspending, stabilizing, or dispersing agents. In one embodiment of a kit, the active ingredient is provided in dry (i.e., powder or granular) form for reconstitution with a suitable vehicle (e.g., sterile pyrogen free water) prior to administration of the reconstituted composition.

The kit may comprise pharmaceutical compositions prepared, packaged, or sold in the form of a sterile aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other formulations which are useful include those which comprise the active ingredient in microcrystalline form, in a liposomal preparation, or as a component of a biodegradable polymer system.

In certain embodiments, the kit comprises instructional material. Instructional material may include a publication, a recording, a diagram, or any other medium of expression which can be used to communicate the usefulness of the device or implant kit described herein. The instructional material of the kit of the invention may, for example, be affixed to a package which contains at least one instruments which may be necessary for the desired procedure. Alternatively, the instructional material may be shipped separately from the package, or may be accessible electronically via a communications network, such as the Internet.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, are not to be construed as limiting in any way the remainder of the disclosure.

### Example 1: Loaded Polymers for Treatment and Prevention of Autoimmune Disorders

The data presented herein demonstrates that PFK15 and bc2 antigen when delivered together in a polymeric particle format leads to generation of adaptive responses in vivo. Moreover, sustained delivery of PFK15 and antigen can be achieved using novel biomaterials that themselves are non-activating and can lead to suppression of DC activation.

paKG Polymers are Generated from Alpha-Ketoglutarate (αKG) and are Capable of Releasing αKG in a Sustained Manner.

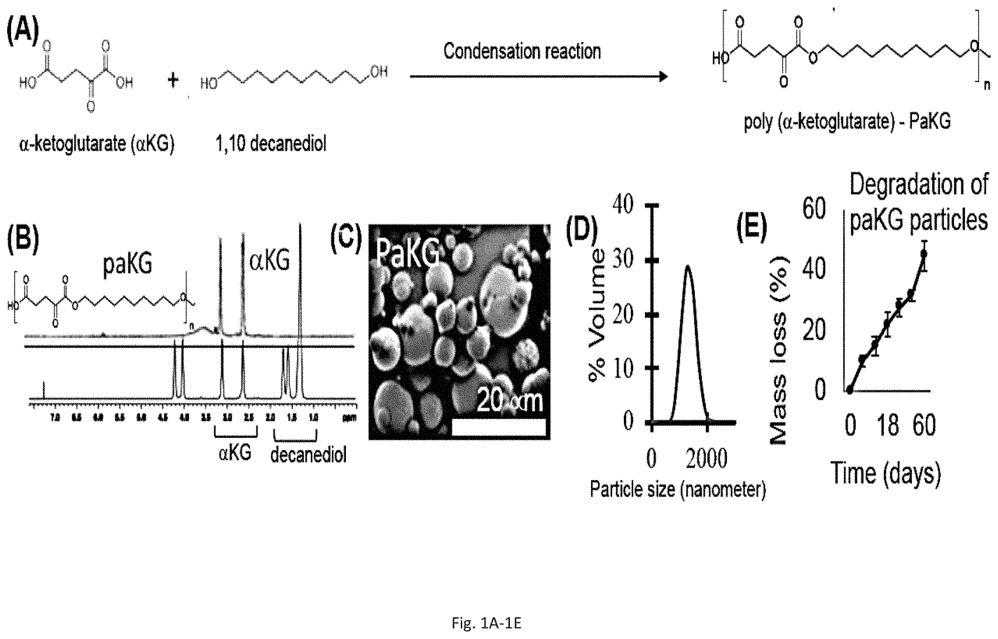

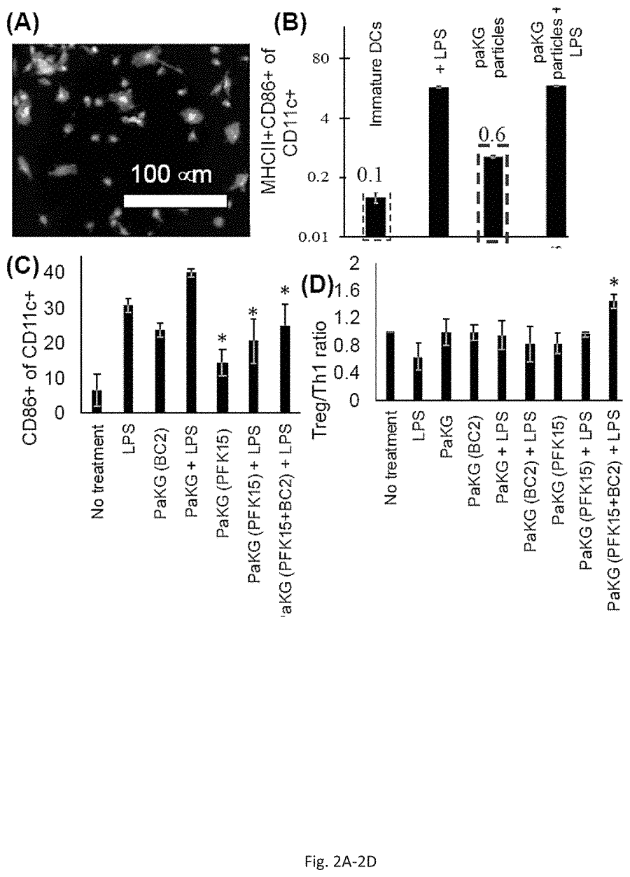

In order to develop a drug delivery vehicle that does not activate dendritic cells (DCs) and is capable of delivering both an antigen and an immunosuppressive agent, paKG polymers were synthesized from the monomers αKG and 1,10 decanediol. αKG is known to favor M2 (suppressive) phenotype rather than M1 (activated) phenotype in macrophages. However, delivery of αKG to modulate the metabolism of immune cells is non-trivial, as this molecule gets metabolized quickly, and will diffuse away from the injection site, and thus will need to be provided via multiple injections, which is prohibitive for RA. A condensation reaction between αKG and 1,10 decanethiol was utilized to generate paKG polymers, which are degradable via hydrolysis and provide sustained release of αKG (FIG. 1A, FIG. 1).

Moreover, polymeric particles were generated out of these polymers using a water-oil emulsion with dichloromethane as the oil phase and water as the aqueous phase. The average size of these particles was determined to be 1 μm using scanning electron microscopy and dynamic light scattering (FIG. 1C, FIG. 1D). Moreover, the particles degrade slowly in phosphate buffered saline solution over 60 days losing approximately 50% of their mass (FIG. 1E) and release αKG in a sustained manner (FIG. 1B—representative 1H NMR—day 10 release shown). These data indicate that paKG were generated, which can release αKG from polymeric particles.

paKG Release PFK15 in a Sustained Manner.

Blocking glycolysis can prevent development of RA symptoms, however, these are systemic treatments, which lead to side-effects. Therefore, technologies have been developed to locally block metabolism of DCs, which can then generate a tolerogenic profile in DCs and lead to antigen-specific T-cell suppression. In order to reduce the risk associated with this method, paKG particles were designed to release PFK15 to block glycolysis in DCs. Specifically, water-oil emulsion techniques were used to encapsulate PFK-15 (hydrophobic drug).

Another set of polymeric particles were generated using water-oil-water emulsion encapsulating bovine collagen type II (bc2—antigen) in the presence of PFK15 in the particles. Percentage encapsulation efficiency (EE) and percentage loading (μg of drug per mg of particles) for bc2, and PFK15 was determined using BCA assay, and absorbance spectrophotometer (plate reader), respectively (Table 1). Moreover, the release of PFK15 were studied using a plate reader, and it was determined that the polymeric particles released the drug in a sustained manner over a period of 30 days.

TABLE 1
Percentage encapsulation efficiency (EE) and percentage loading

| | % EE | % loading (μg/mg) |
|---|---|---|
| bc2 | 60 | 3 |
| PFK15 | 68 | 1.7 |

To generate immunosuppressive DCs, these cells should be able to express antigens and at the same time generate low levels of activating signals (co-stimulatory molecules and cytokines). In order to achieve this response, intracellular delivery of glycolytic inhibitor was used to generate an immunosuppressive DC phenotype (MHCII⁺CD86^{Lo}IL-10⁺IL-12p70^{Lo}) DCs were differentiated from bone marrow of C57BL/6j using a 10 day-protocol (>90% purity). Next, paKG particles that are designed to encapsulate and release glycolytic inhibitor PFK15 and designed to be phagocytosed were utilized for this purpose. After a 2-hour incubation of rhodamine (representative drug molecule) encapsulated paKG particles with DCs, it was observed that the bone marrow derived DCs were able to phagocytose these particles effectively (FIG. 2A).

Next, in order to assess whether the particles, which are capable of releasing αKG induce activation in bone marrow derived DCs, these cells were cultured with paKG for 24 hours, and the cells were then stained for CD1 Ic, CD86 and MHC-II. It was observed that the paKG particles marginally increased activation in DCs, which was not significantly different than immature DCs (FIG. 2B). Moreover, the IL-10 and IL-12p70 expression demonstrated that paKG+LPS group expressed equivalent levels of IL-10 as the positive control of LPS only, but expressed 2.5-fold lower levels of IL-12p70 as compared to LPS only group. These data indicate that paKG particles alone can have immunosuppressive quality to them.

In order to evaluate the ability of paKG particles delivering PFK15 and the antigen bc2 to prevent the activation of DCs, particles encapsulating [PFK15] or [PFK15+bc2] or [bc2] were generated. These particles were then incubated with DCs at 0.1 mg/mL concentration for 24 hours and the surface expression of CD11c, and CD86 was evaluated using flow cytometry. It was observed that even in the presence of LPS, paKG particles delivering PFK15 or PFK15+BC2, significantly reduced the activation of DCs as compared to the paKG+LPS condition. These data indicate that paKG polymeric particles delivering PFK15 and bc2 generated immune suppressive phenotype in DCs (FIG. 2C).

Next, in order to evaluate whether the modulation in DC responses can be also observed in adaptive T-cell responses, paKG particles encapsulating different factors in the presence or absence of LPS were added to a syngeneic mixed lymphocyte reaction. No treatment was used as a negative control and LPS treatment only was used as a positive control. Specifically, 20,000 bone-marrow derived DCs were cultured with 200,000 CD3+ T-cells (isolated from spleen of C57BL/6j mice and magnetically separated) for 48-72 hours in the presence of particles. The T-cells were then stained for CD4, CD25, Tbet, live/dead stain, and Foxp3, and analyzed using flow cytometer for Th1 (CD4⁺Tbet⁺), and Tregs (CD4⁺CD25⁺Foxp3⁺). It was observed that the paKG particles delivering PFK15 along with BC2 in the presence of LPS significantly upregulated the TREG/Th1 ratio as compared to all the other conditions. These data indicate that the intracellular delivery of PFK15 and the antigen bc2 via paKG particles in DCs induced a tolerogenic phenotype, which then lead to tolerogenic adaptive responses in vitro.

paKG Polymeric Particles Delivering PFK15 and Antigen bc2 Modulates CIA in DBA/1j Mice

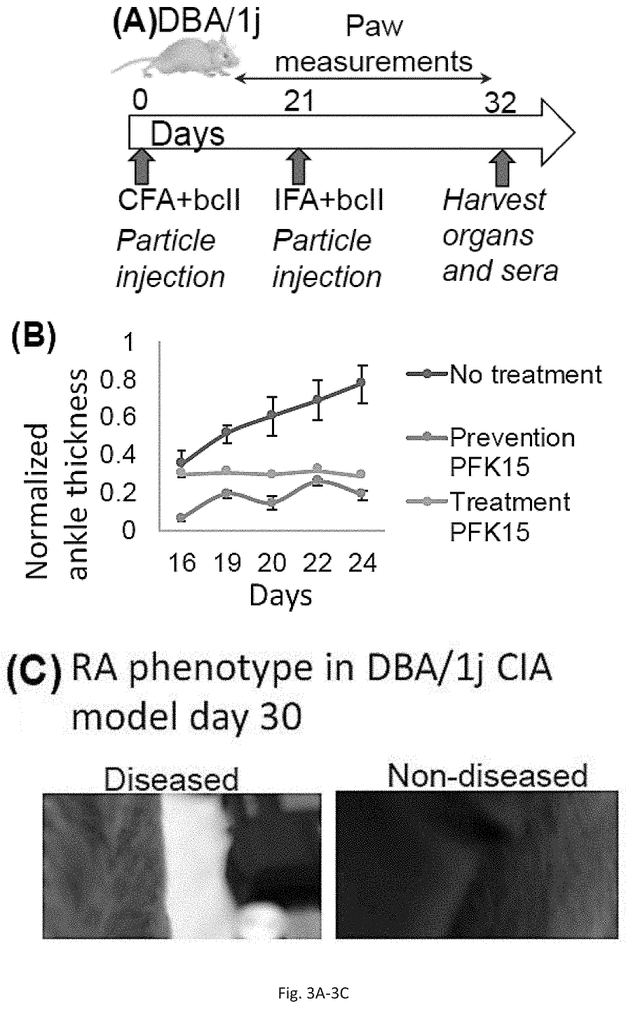

In order to determine if the metabolic inhibition of immune cells can modulate responses in vivo, the collagen induced arthritis (CIA) model in DBA/1j mice was utilized (FIG. 3A). Specifically, DBA/1j mice were injected with CFA+bc2 emulsion on day 0, and on day 21 booster IFA+bc2 was provided to the mice. Mice were also injected with 1 mg of particles encapsulating PFK15+bc2 subcutaneously in the back of the mice on two sides, on days 0, 16, and 21 for prevention studies, and on day 16 and 21 for treatment studies. Moreover, ankle thickness measurements were performed using Vernier calipers from day 16 to day 32 every other day. It was observed that with PFK15+bc2 particles thickening of the ankle was significantly lower than the no treatment control, and the PFK15 treatment group (FIG. 3B). These data indicate that particles locally delivering PFK15+bc2 may be a viable strategy to prevent RA progression.

paKG Particles Delivering PFK15 and bc2 In Vivo Upregulate Antigen Specific Anti-Inflammatory Responses

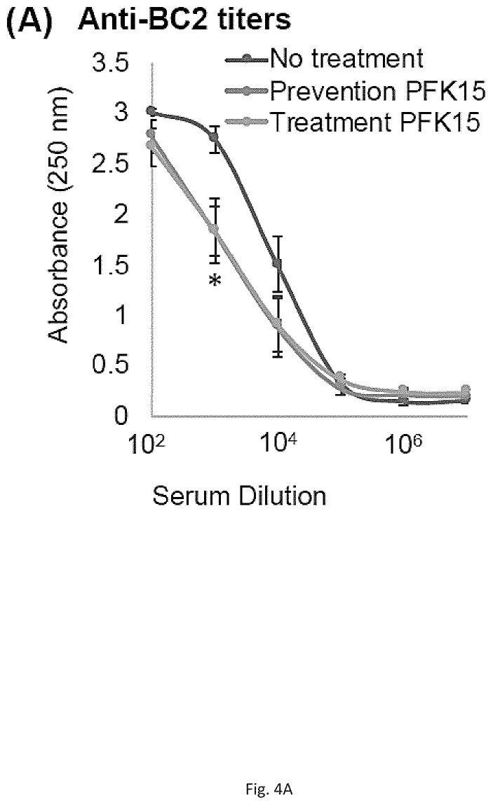

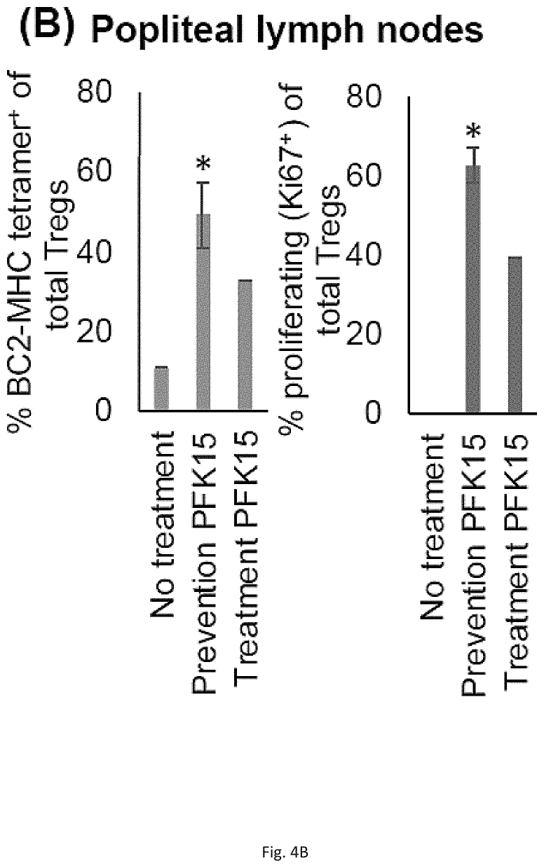

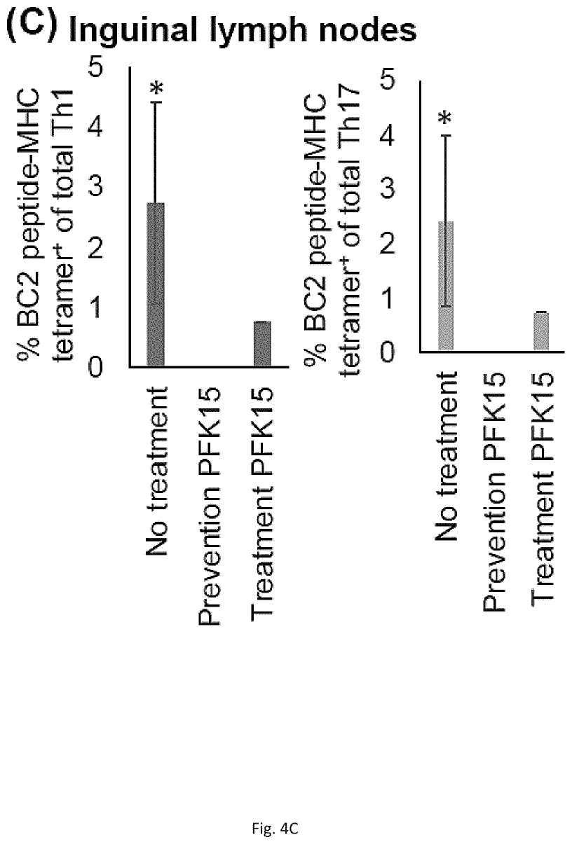

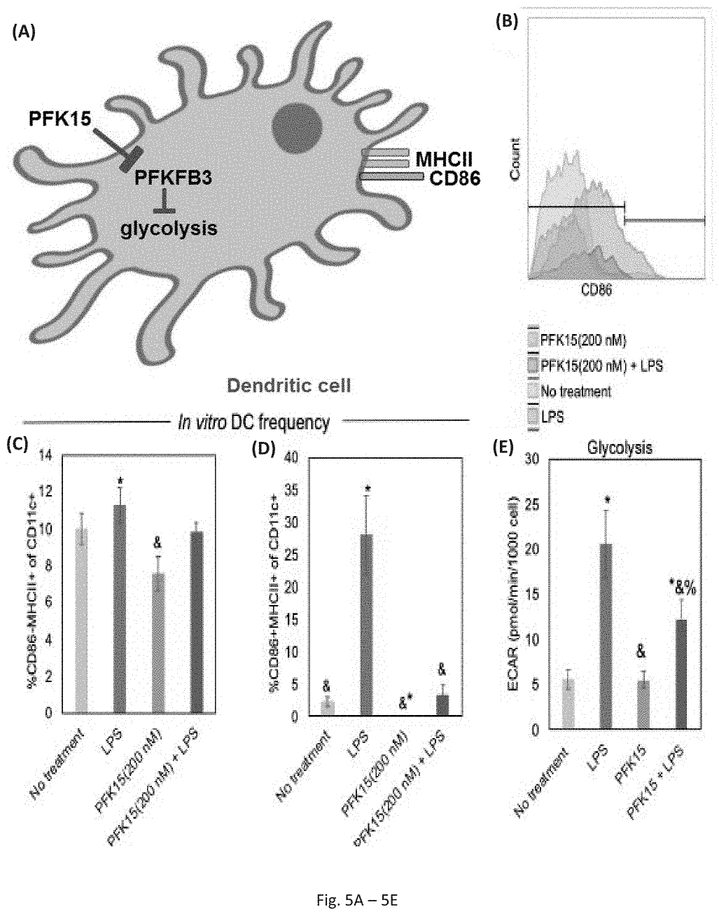

In order to determine whether inhibition of local glycolysis in the presence of the antigen induces antigen-specific anti-inflammatory responses, mice were injected with PFK15+bc2 in DBA/1j mice on days 0, 16, and 21 for prevention studies, and on day 16 and 21 for treatment studies (n=5 per group). These mice were then sacrificed on day 30, and blood was harvested. Serum was isolated from the blood and anti-bc2 IgG titers were determined using ELISA. It was determined that in both the prevention and the treatment groups the antibody titers against bc2 were significantly lower than the no treatment group. This data indicates that the co-delivery of PFK15 and the antigen may have a role in preventing auto-antigenic antibodies (FIG. 4A).

On day 30, inguinal lymph nodes, popliteal lymph nodes, and spleen were also harvested from these mice. The cells were obtained from these organs and stained for surface markers CD4, CD8, CD25, CD44, MHC-tetramer (against bc2 peptide I-A(q) bovine collagen II 271-285 GEPGIAGFKGEQGPK (SEQ ID NO: 1)), and intracellular markers, Ki67, Tbet, RORγt and Foxp3. These cells were analyzed for the presence of Th1, Th17, Tregs, bc2-specific Tregs, proliferating Tregs, and proliferating Th17 cells using flow cytometer. It was observed that the prevention group significantly upregulated antigen-specific Treg population and total proliferating Tregs in the popliteal lymph nodes (FIG. 4B). Moreover, the frequency of Tregs in the popliteal lymph nodes was 10-fold higher in the prevention group as compared to the no treatment group. The frequency of Tregs in the popliteal lymph node was not significantly different between PFK15 the treatment group and the no treatment group. The prevention and the treatment groups both up-regulated the proliferation (Ki67+) of Tregs in the popliteal lymph nodes (FIG. 4B). Notably, in the inguinal lymph node, no treatment group and PFK15 treatment groups had elevated levels of antigen-specific Th1 and Th17 pro-inflammatory cells, whereas in the prevention group these cells were at undetectable levels (FIG. 4C). Furthermore, increased levels of CD4⁺RORγt⁺CD44⁺, CD4⁺Tbet⁺CD44⁺ were found in the popliteal lymph nodes in the no treatment group as compared to the PFK15 prevention groups, which indicates that there was a reduction of memory phenotype in the PFK15 prevention group. Lastly, T-cells isolated from lymph nodes and spleen were cultured for 48 hours in the presence of bc2 antigen (20 μg/mL), IL-2, anti-CD3 and anti-CD28 ex vivo in order to study the antigen-specific recall reaction. It was observed that in the no treatment group, a significantly higher (˜2-fold) frequency of Th1 and Th17 cell proliferation was observed, as compared to the prevention group. There was no significant difference between the PFK15 treatment group and the no treatment group in the recall reactions.

PFK15 Blocks Glycolysis and Reduces Co-Stimulatory CD86 Expression in Bone Marrow-Derived DCs In Vitro

Reduction in CD86 expression by inhibiting glycolysis in DCs can affect pro-inflammatory DC activation in the presence of lipopolysaccharide (LPS), which is used to mimic inflammation in vitro (FIG. 5A). Therefore, it was tested if PFK15 by itself can block glycolysis in bone marrow-derived DCs (BMDCs) in the presence or absence of LPS. To assess this, the extracellular acidification rate (ECAR) of BMDCs was determined by extracellular flux assays. BMDCs treated with LPS only and no treatment were used as controls. It was observed that, in the presence of LPS, PFK15 significantly lowered glycolysis (ECAR) in BMDCs by 60% as compared to LPS alone. Interestingly, when compared to no treatment, PFK15 without LPS did not significantly reduce ECAR in BMDCs (FIG. 5E). These data show that in the presence of LPS, PFK15 can block glycolysis in BMDCs, whereas, in the absence of LPS, PFK15 does not affect BMDC glycolysis.

Next, it was assessed if the changes in BMDC glycolytic rates can affect surface co-stimulatory CD86 expression in BMDCs, using flow cytometry. In this study, pro-inflammatory DCs were defined as the frequency of CD86⁺MHCII⁺ of CD11c⁺ cells and anti-inflammatory DCs were defined as CD86⁺MHCII⁺ of CD11c⁺ cells. It was observed that PFK15, with and without LPS, reduced CD86 expression in vitro in CD11c⁺ cells when compared to LPS and no treatment (FIG. 5B). It was also observed that PFK15, in the presence of LPS, significantly decreased the frequency of CD86⁺MHCII⁺ of CD11c⁺ BMDCs by approximately 8-fold when compared to LPS. Similarly, in the absence of LPS, PFK15 also significantly decreased the frequency of the pro-inflammatory BMDC populations as compared to no treatment (FIG. 5C). Interestingly, PFK15 by itself, with and without LPS, did not significantly decrease anti-inflammatory BMDC populations as compared to LPS and no treatment (FIG. 5D). Overall, these data suggest that, PFK15 can suppress CD86 expression in BMDCs in vitro and can also block glycolysis in BMDCs in the presence of LPS.

PFK15 is Encapsulated in αKG-Based MPs to Simultaneously Deliver αKG and PFK15 to DCs

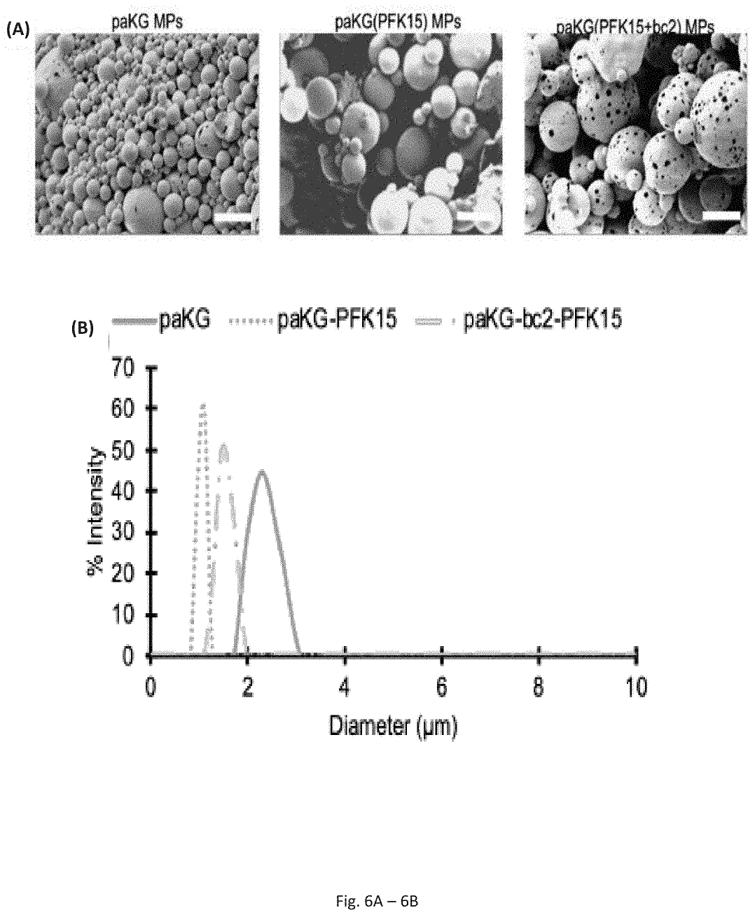

PFK15, when delivered systemically, can induce cytotoxic effects and inhibit glycolysis, or the function of cells that partake in generating host immunity against foreign and invading pathogens. Therefore, to minimize the effect on host immunity while promoting a controlled delivery of PFK15 to DCs, PFK15 would need to be contained in a carrier for DC phagocytosis and intracellular delivery. Interestingly, a metabolite-based MP, that was generated from the immunosuppressive metabolite and Krebs cycle intermediate, αKG, can associate with DCs and release αKG in a sustained manner as the MP degrades. Importantly, this technology can be designed to encapsulate desired materials for delivery. Therefore, inhibiting glycolysis (using PFK15) while simultaneously providing αKG (from metabolite-based MP) may allow for Krebs cycle-mediated cellular energy and OXPHOS in DCs. This approach may potentially inhibit pro-inflammatory DC function and favor anti-inflammatory DC phenotypes. To test this, PFK15 was encapsulated in paKG MPs (termed paKG(PFK15) MPs) using water in oil emulsion techniques. The morphology and heterogeneity of paKG and paKG(PFK15) MPs were confirmed by scanning electron microscopy (SEM). Both MPs displayed spherical shapes with a smooth surface morphology (FIG. 6A). The average size of paKG MPs without PFK15 was 2.3±0.3 μm and paKG(PFK15) MPs was 1±0.1 μm, as determined by dynamic light scattering (DLS) (FIG. 6B). The percentage encapsulation efficiency of PFK15 within paKG MPs was 68±17% and the percentage loading efficiency was 2±1 μg/mg of paKG MPs.

Furthermore, paKG MP endotoxin levels were determined to be <0.01 EU/mL, therefore, demonstrating that the MPs have low levels of endotoxins. Overall, these data suggest that paKG MPs may be a feasible approach to locally deliver PFK15.

BMDCs Phagocytose paKG-Based MPs In Vitro

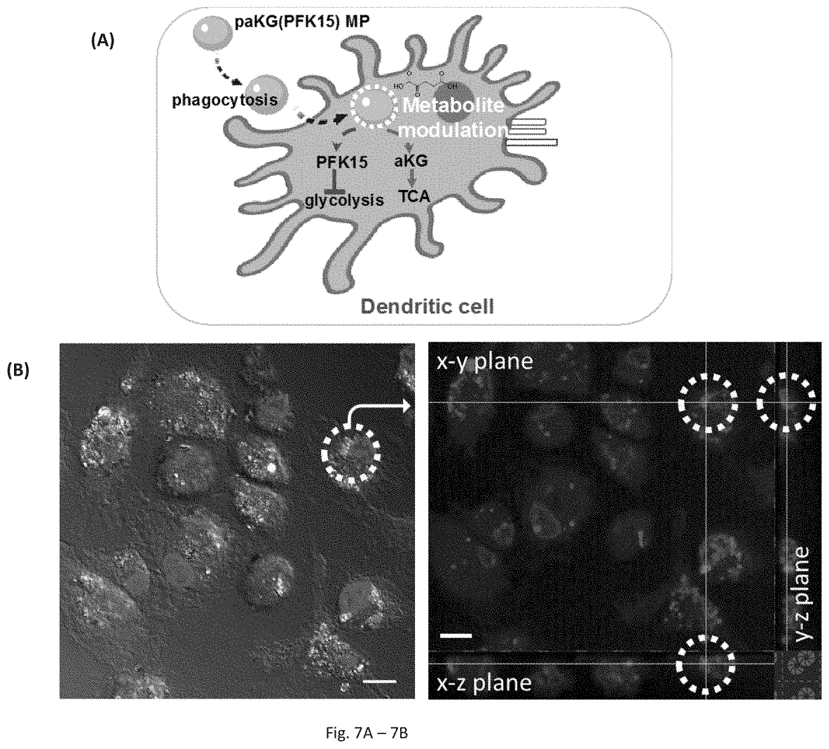

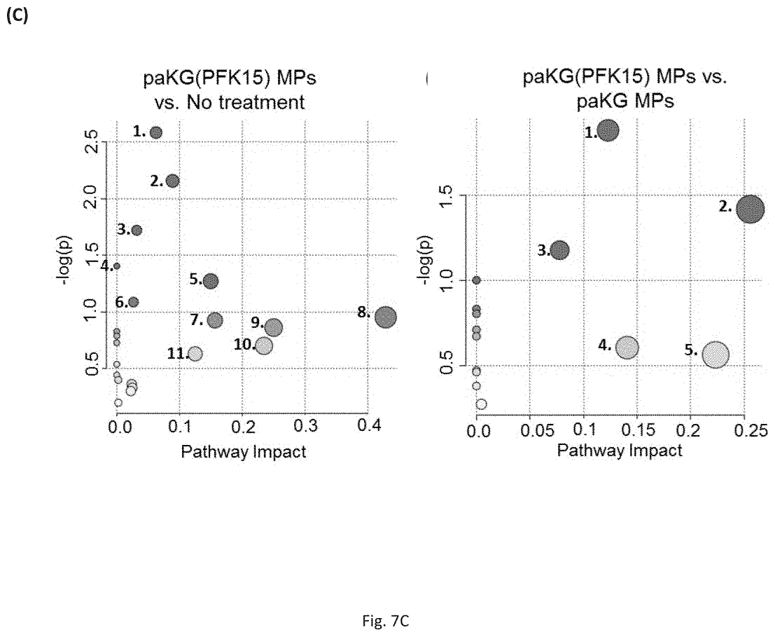

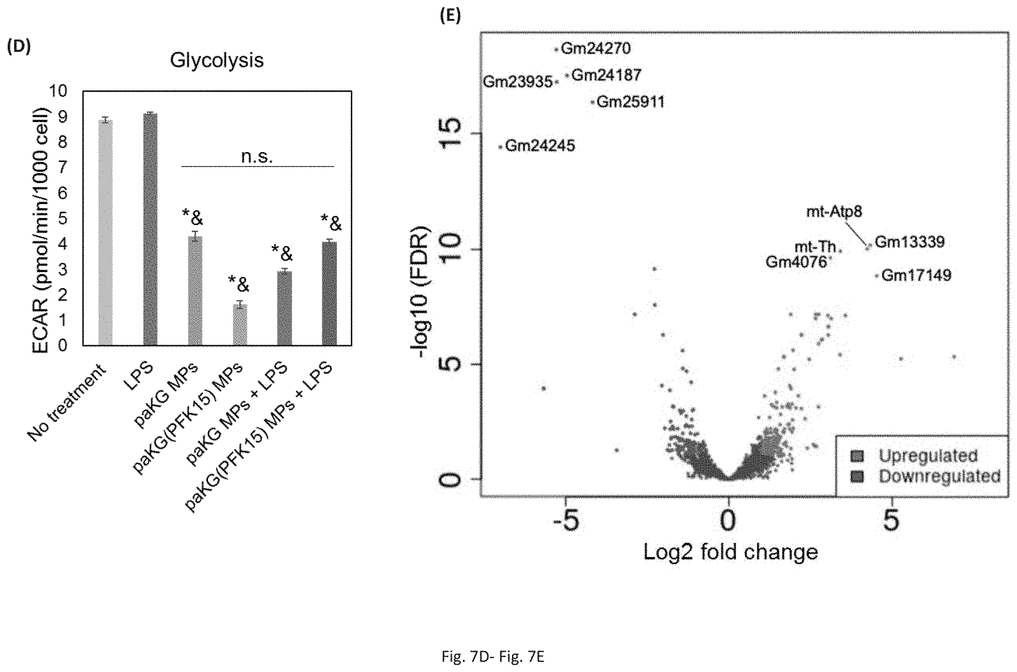

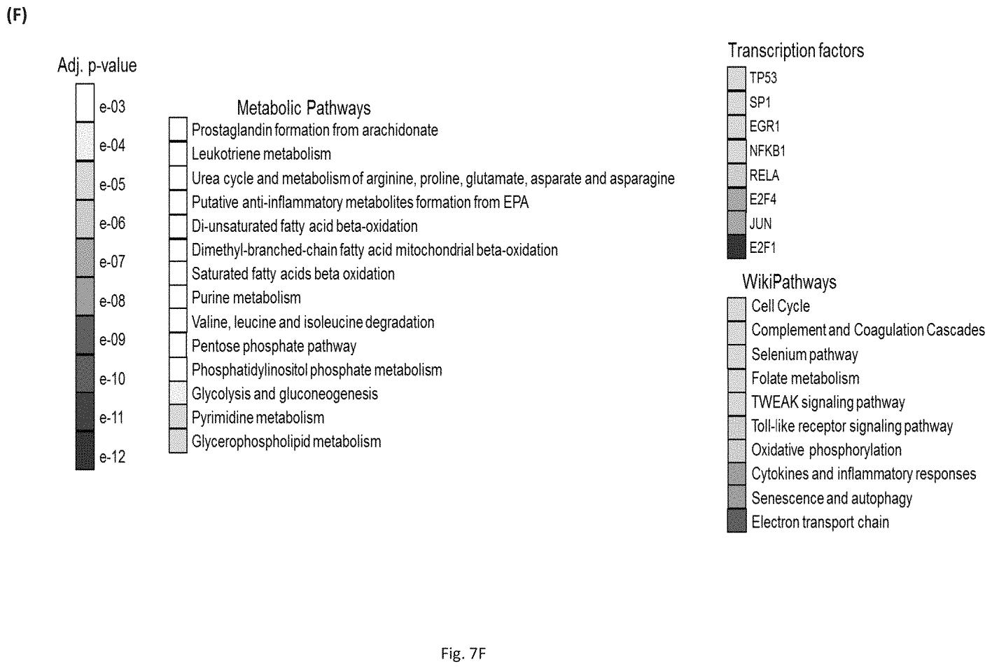

In order for paKG(PFK15) MPs to modulate DC function, paKG MPs need to be phagocytosed by DCs to deliver PFK15 intracellularly (FIG. 7A). Confocal imaging demonstrated that paKG MPs encapsulating rhodamine 6G, a fluorescent dye, can be internalized by BMDCs as observed by images containing magenta (paKG(rhodamine 6G) MPs) and blue (nucleus of BMDCs) (FIG. 7B). Additionally, live/dead viability stains, as observed by flow cytometry, confirmed that the 0.01 mg/mL concentration of paKG-based MPs that were added to the incubated BMDC cultures were non-toxic. In all, this data demonstrates that BMDCs are able to phagocytose paKG-based MPs in vitro.

paKG(PFK15) MPs Block BMDC Glycolysis and Alters Metabolic Pathways

Since metabolites can control DC function, and since it was observed that PFK15 alone can modify the glycolytic rate and activation of pro-inflammatory BMDCs, the effect of delivering PFK15 in paKG MPs on the intracellular metabolic profile of BMDCs needs to be investigated. Although the data demonstrates that PFK15 alone can modify the glycolytic rate of BMDCs, it is still yet to be determined if the glycolytic rate can still be modified when PFK15 is delivered via paKG MPs. To test this, extracellular flux assays were used, and pre-activated DCs with LPS and no treatment were used as controls. It was observed that, paKG(PFK15) MPs, significantly decreased glycolysis (ECAR) in pre-activated BMDCs by approximately 2-fold, as compared to LPS alone. Similarly, paKG(PFK15) MPs, without LPS treatment, also significantly decreased glycolysis by approximately 5-fold, as compared to no treatment (FIG. 7D). Together, these data demonstrate that PFK15, when contained and delivered by paKG MPs, block glycolysis in BMDCs.

Next, the ability of paKG(PFK15) MPs to impact metabolite accumulation and metabolic pathways, other than glycolysis, in BMDCs was investigated. Using liquid chromatography mass spectrometry (LC-MS/MS), it was determined that the steady release of PFK15 from paKG MPs significantly affected 28 metabolites out of the 299 intracellular metabolites tested, as compared to no treatment. Additionally, 34 out of the 299 intracellular metabolites tested were affected when comparing paKG(PFK15) MPs to paKG MPs. The Kyoto Encyclopedia of Genes and Genomes (KEGG) database and metabolite intensities were utilized to identify the metabolic pathways that were the most impacted and an interactive visualization framework was constructed to visualize the relevant pathways. The metabolic pathways that had an impact value >0.1 or −log (p)>10 were determined as the most relevant pathways. When comparing paKG(PFK15) MPs to no treatment, 11 metabolic pathways in BMDCs were significantly impacted with a ˜2-5 fold alteration in metabolites within amino acid metabolism such as alanine and aspartate metabolism; tryptophan; alanine, aspartate and glutamate metabolism; taurine and hypotaurine; and cysteine and methionine metabolism. Additionally, metabolites in carbohydrate metabolism, such as the citrate cycle; glycolysis/gluconeogenesis; pyruvate metabolism; butanoate metabolism; ascorbate and alderate metabolism; and pentose and glucuronate interconversions were also impacted by ˜2-5 fold when comparing paKG(PFK15) MPs to no treatment, as well as, pathways involved in metabolism of cofactors and vitamins, such as nicotinate and nicotinamide metabolism (FIG. 7C). Furthermore, when comparing paKG(PFK15) MPs to paKG MPs, the metabolic pathways in BMDCs that were significantly impacted were pathways involved amino acid metabolism such as histidine metabolism; glutathione metabolism, arginine and proline metabolism; lysine degradation; alanine and aspartate and glutamate metabolism (FIG. 7C). In all, these data demonstrate that paKG(PFK15) MPs modulate BMDC metabolism by directly changing the intracellular metabolite profile.

paKG(PFK15) MPs Suppress Genes Encoding Pro-Inflammatory Responses in BMDCs

In order to understand the relation between the phenotypic changes observed in BMDCs to the cellular signaling pathways and differentially expressed genes within modified BMDCs, RNA-seq on treated BMDCs was performed. When comparing paKG(PFK15) MPs vs. no treatment, volcano plots demonstrated a downregulation in miRNA associated genes Gm24270 (log 2 fold change: −5), Gm24187 (log 2 fold change: −5), Gm23935 (log 2 fold change: −5), Gm25911 (log 2 fold change: −4), and Gm24245 (log 2 fold change: −7). Additionally, an upregulation was observed in protein coding (mt-Atp8; log 2 fold change: 4), Mt-tRNA (mt-Th; log 2 fold change: 3), and pseudogenes that are unprocessed (Gm13339; log 2 fold change: 4) and processed (Gm4076 (log 2 fold change: 3) and Gm17149 (log 2 fold change: 4)). Furthermore, these volcano plots highlighted an upregulation and enrichment of the electron transport chain (adj. p-value: 3.6e-15), oxidative phosphorylation (adj. p-value: 1.1e-10), and mRNA processing (adj. p-value: 5.6e-05) when comparing paKG(PFK15) MP treated BMDCs to no treatment (FIG. 7E). Moreover, pathways such as the metabolic pathway, transcription factor pathways and wikipathways demonstrated an inverse relation in gene expression when comparing paKG(PFK15) MP treated BMDCs to no treatment. These gene sets were associated with immune response pathways that involved increasing anti-inflammatory metabolite formation and fatty acid oxidation, and decreasing pro-inflammatory associated genes related to glycolytic processes, toll-like receptor signaling and cytokines and inflammatory responses. Of particular relevance, paKG(PFK15) MPs upregulated genes related to anti-inflammatory responses, such as genes encoding the electron transport chain, TGFβ signaling pathway, and glutathione metabolism. Importantly, paKG(PFK15) MPs downregulated genes related to pro-inflammatory responses, such as genes encoding Tnf, pfkfb3, HIF1a and several genes associated with the IL-6 signaling pathway (FIG. 7F). In all, RNA-seq data demonstrated that paKG(PFK15) MPs modulate immune responses in BMDC's by altering the expression of genes that are involved in pro-inflammatory immune processes.

The Self-Presenting CIA Antigen, bc2, is Encapsulated within paKG(PFK15) MPs and Normalizes bc2-Driven Paw Thickness in CIA Mice

Thus far, it has been determined that PFK15 can be contained and when delivered to BMDCs via paKG MPs, can decrease the glycolytic rate and upregulate anti-inflammatory associated metabolites and metabolic pathways. Notably, anti-inflammatory DCs (low CD86 expression) have also been shown to generate antigen specific immune suppression when expressing an antigen. Therefore, the ability of paKG(PFK15) MPs to generate antigen-specific immune suppression via glycolytic inhibition for the treatment of autoimmune diseases was tested. Specifically, a formulation consisting of PFK15 and bc2 (antigen) was generated and tested in CIA mouse model of RA. Bc2 is the antigen that triggers RA symptoms in the CIA mouse model, thus, retraining the immune system to tolerate bc2 may reinstate immune tolerance in CIA mice. This may be achieved in vivo by delivering bc2 to anti-inflammatory DCs that sustain energy from the Krebs cycle (attained by delivering aKG) and, concurrently, have a reduced energy reliance on glycolysis (attained by delivering PFK15). Therefore, PFK15+bc2 was encapsulated in paKG MPs (termed paKG(PFK15+bc2) MPs) using water-oil-water emulsion techniques. This will potentially allow the formulation to simultaneously deliver αKG, PFK15, and bc2 to the same DC as the MP degrades. SEM analysis demonstrated that paKG(PFK15+bc2) MPs were more porous than paKG and paKG(PFK15) MPs, however all three paKG-based MPs were spherical and heterogeneous (FIG. 6A). DLS determined the average size of the paKG(PFK15+bc2) MPs to be 1.5±0.2 μm (FIG. 6B). The percentage encapsulation efficiency of bc2 was 60% (wt/wt) and the percentage loading efficiency was 3 □g/mg of MPs. These data indicate that paKG MPs can encapsulate both PFK15 and bc2.

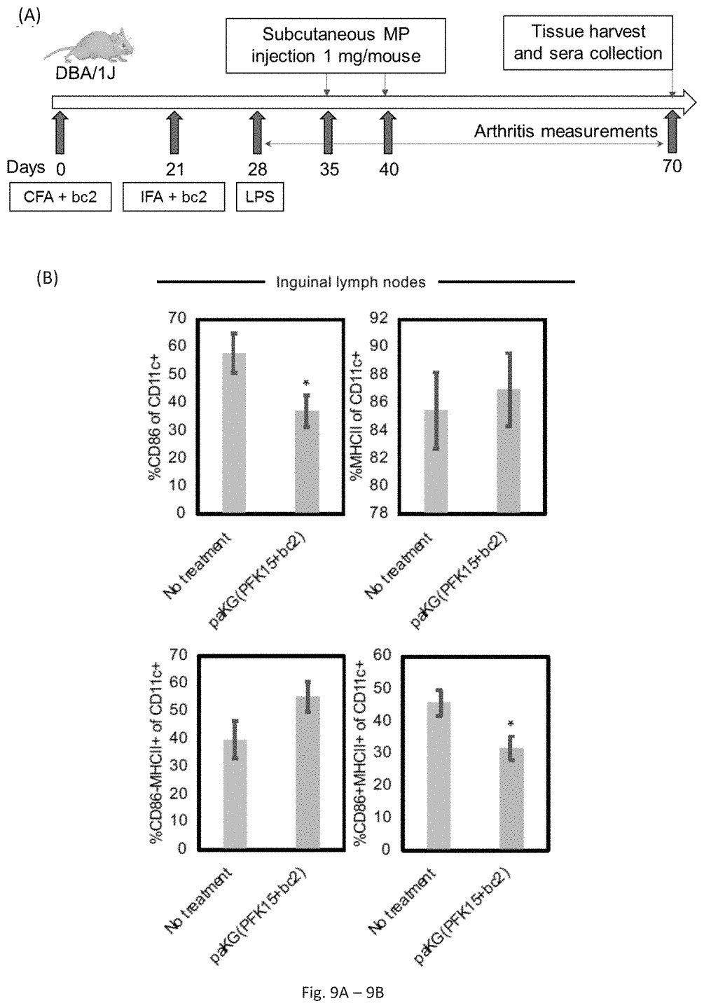

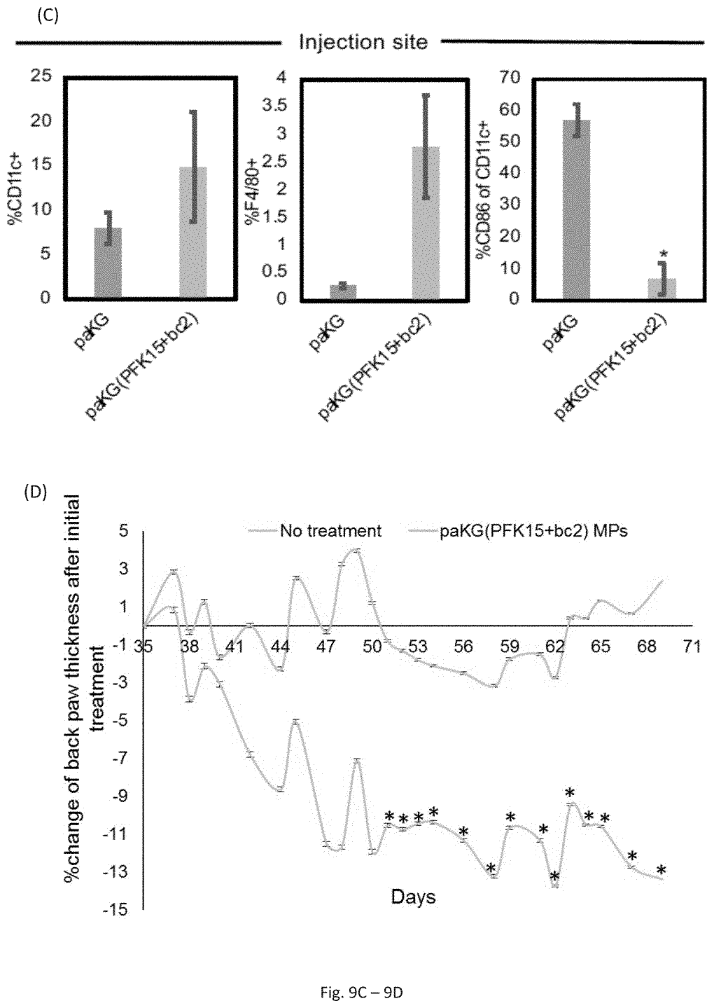

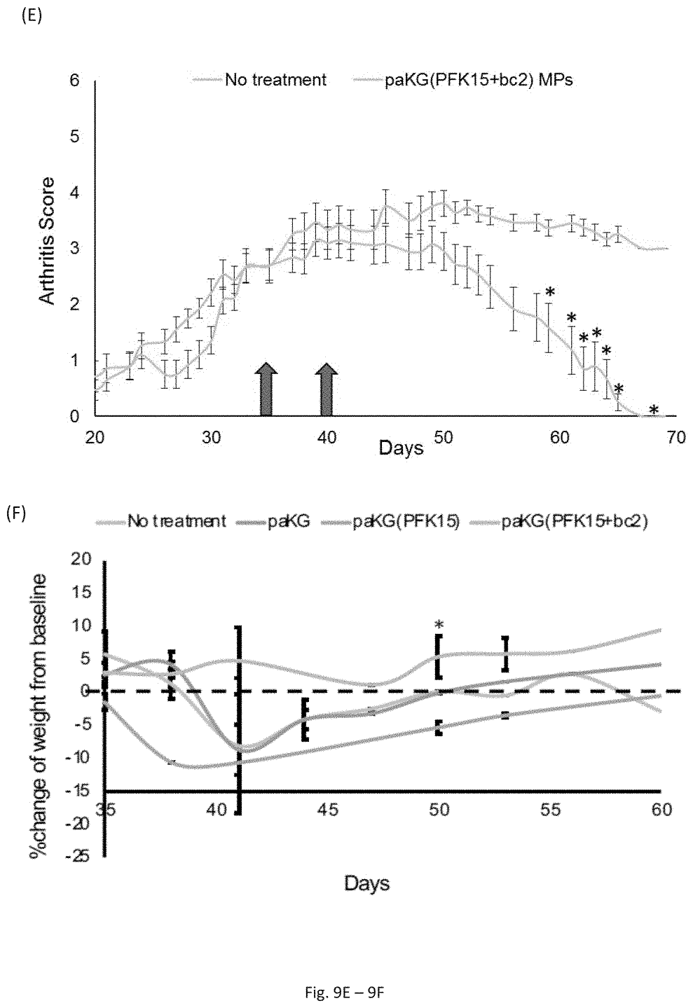

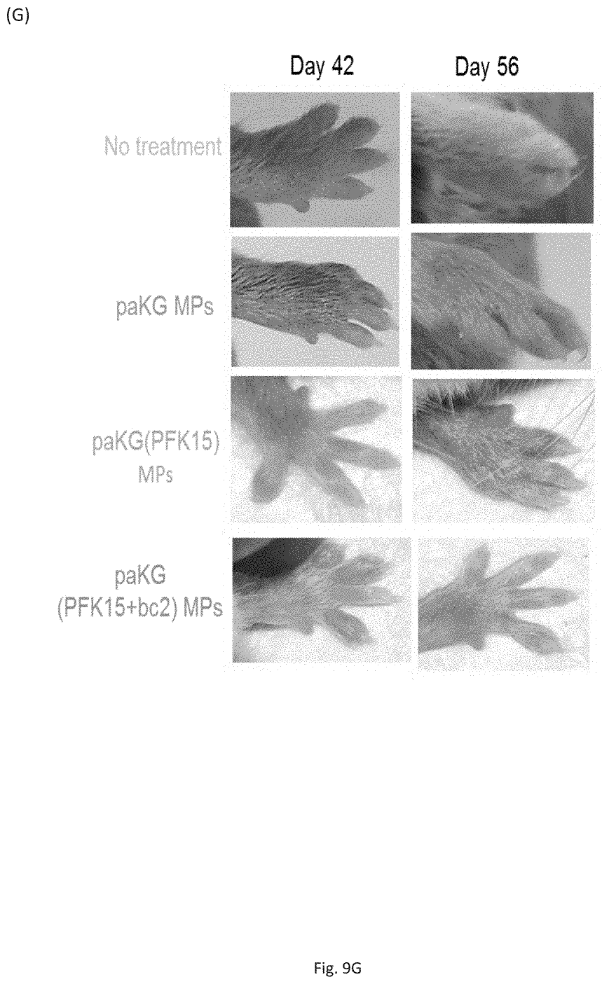

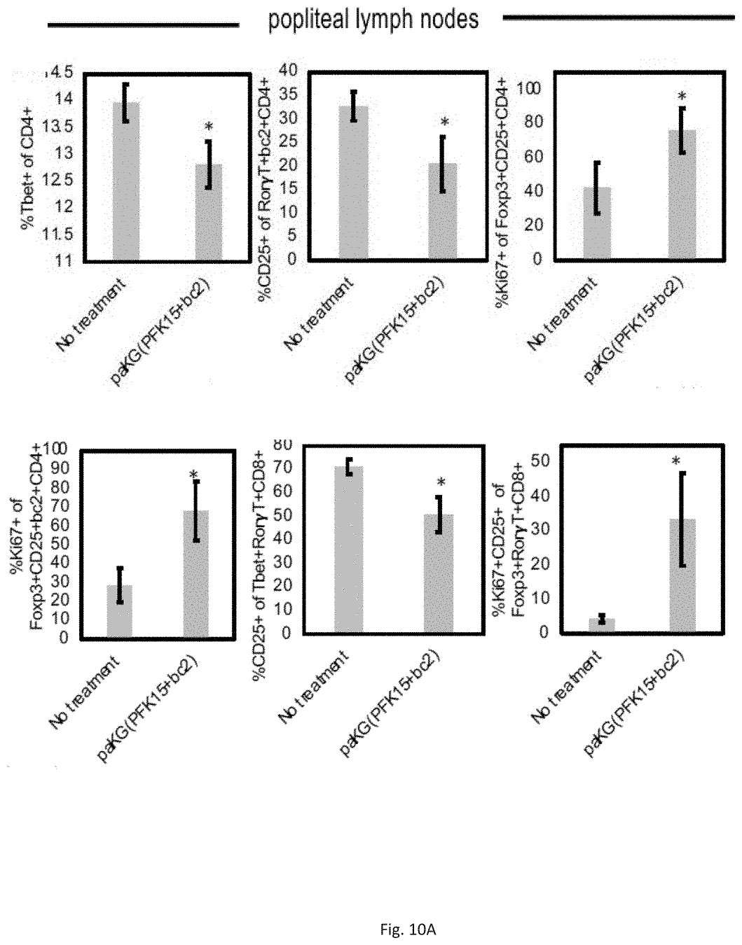

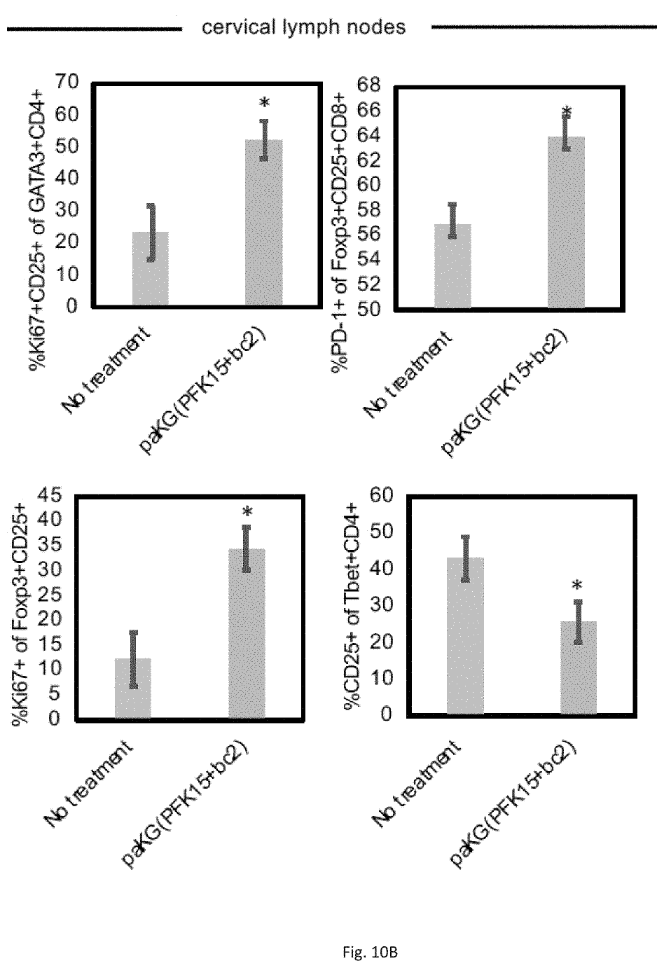

To determine if the metabolic inhibition in the presence of the bc2 antigen can reverse RA symptoms, the CIA model in DBA/1j mice was utilized. The severity of the arthritis was determined by an arthritic score on a scale of 0 to 6. A score of 3 or higher signified moderate or severe arthritis. Mice were administered 1 mg of either empty paKG MPs, paKG(PFK15) MPs, or paKG(PFK15+bc2) MPs subcutaneously near the two hind legs, on day 35 (an average arthritic score of 3) and day 40 (FIG. 9A). Injections of phosphate buffered saline (PBS) were used as a no treatment control. The back paws of the mice were observed to determine the local effects of the disease and treatment, since the treatments were administered near the two hind legs. The front paws of the mice were observed to determine the systemic effects of the disease and treatment. It was determined that the percent change of the back paw thickness of mice treated with paKG(PFK15+bc2) MPs was overall lower than all controls and was consistently less than the no treatment group throughout the study. Specifically, after the day 35 treatment, the percent change of back paw thickness for paKG(PFK15+bc2) MP treated mice was significantly lower than no treatment on days 52 to the end of study measurement, day 69 (FIG. 9D). Moreover, when comparing arthritic scores, it was observed that, after the day 35 treatment, the paKG(PFK15+bc2) MP treated mice had significantly lower arthritic scores than untreated mice on days 59 to the end of study measurement. Importantly, mice treated with paKG(PFK15+bc2) MPs had an arthritic score of 0 starting at day 67 to the end of the study (FIG. 9E). However, when the paKG(PFK15+bc2) MP treated mice to the control mice, the percent change of the back paw thickness for paKG(PFK15) MP treated mice was significantly lower than no treatment as of day 52 to the end of the study. Conversely, mice treated with paKG MPs had significantly increased percentages of back paw thickness when compared to no treatment on days 44, 47,48 and 49 and significantly lower percentages of back paw thickness on days 45 and 58 to the end of the study. Notably, paKG(PFK15) MP treated mice had higher levels of paw inflammation prior to treatment than paKG(PFK15+bc2) and paKG MP treated mice, hence, paKG(PFK15) MP treated mice had a greater percentage change in back paw thickness. (FIG. 13A). However, when observing arthritic scores, mice receiving paKG(PFK15) MPs had increased arthritic scores throughout the study when compared to no treatment, where the arthritic score was significantly higher than no treatment from days 37 to 49 and days 61, 62 and 64 to the end of study. Similarly, mice treated with paKG MPs had significantly higher arthritic scores on days 48 and 49 and days 51 and 52 with an end of study measurement of an arthritic score that was not significantly different than no treatment (FIG. 13B). Moreover, it was observed that the front paws of mice treated with paKG(PFK15+bc2) MPs on day 56 had decreased swelling as compared to no treatment, paKG MPs, and paKG(PFK15) MPs (FIG. 9G), suggesting a systemic response within 3 weeks of the first treatment. These data suggest that local injections of paKG(PFK15+bc2) MPs are a viable strategy to systemically treat RA. Furthermore, the reduction in paw swelling, in the paKG(PFK15+bc2) MP treatment group, indicates that the delivery of PFK15+bc2 via paKG MPs may induce antigen-specific immune suppression via glycolytic inhibition in DCs in a CIA mouse model.

paKG(PFK15+bc2) MPs Decrease the Frequency of CD86⁺MHCII+ BMDC Populations and Subsequent Pro-Inflammatory Cytokine Production In Vitro

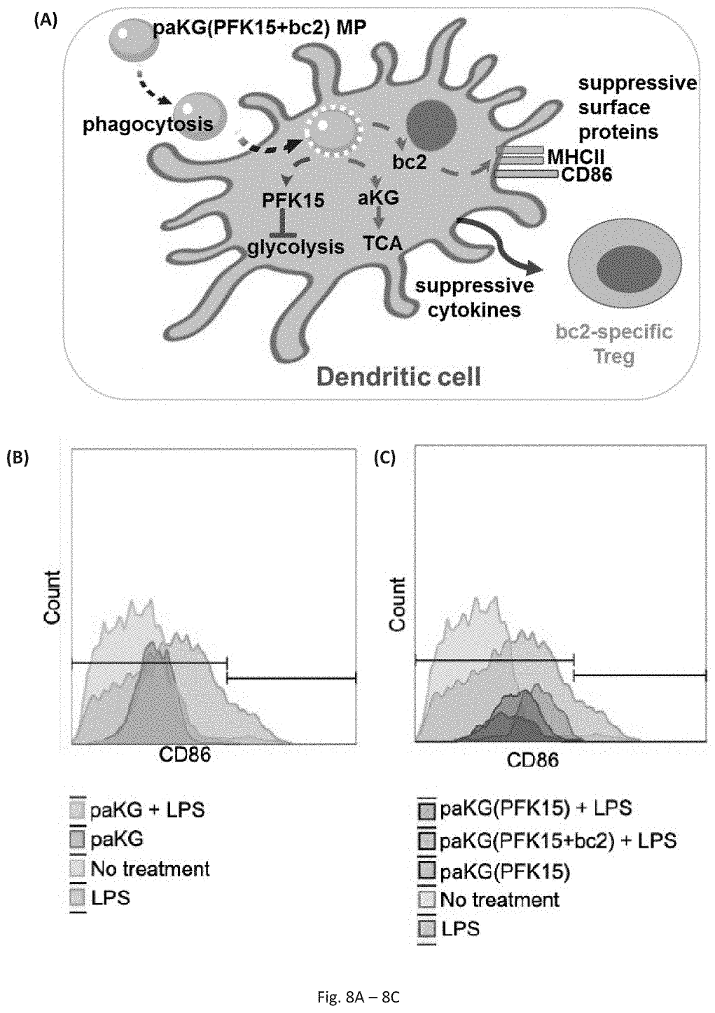

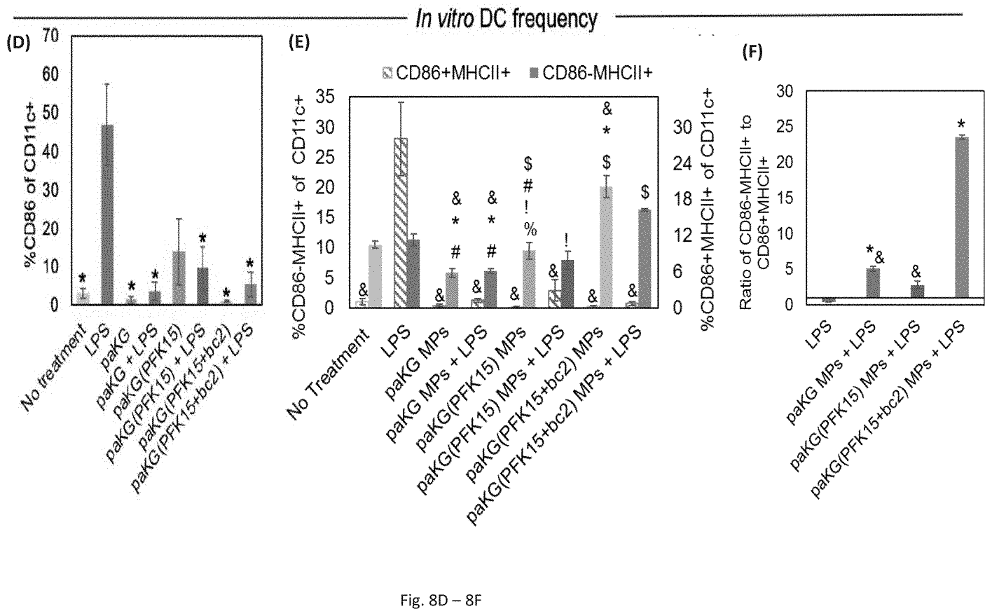

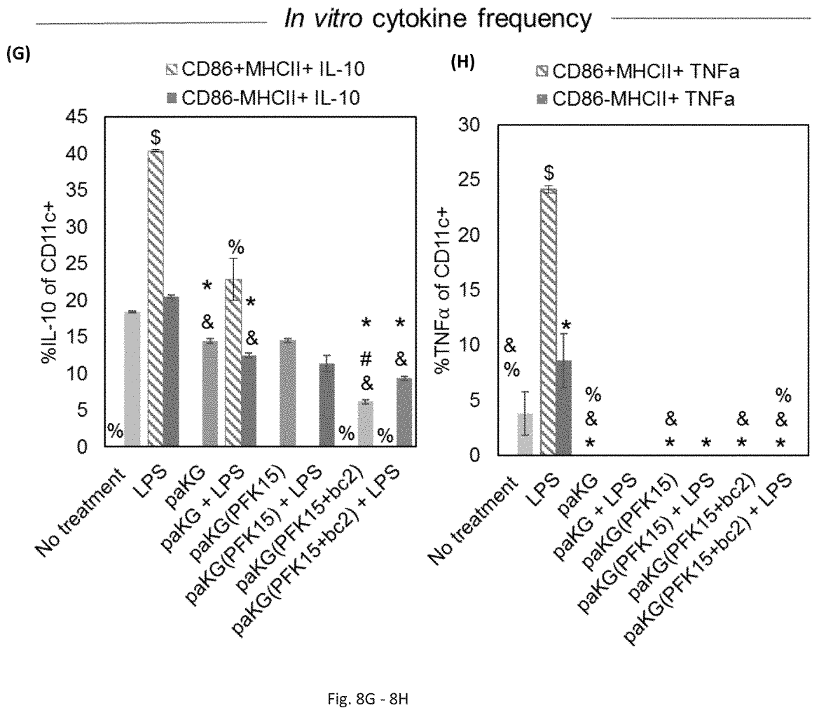

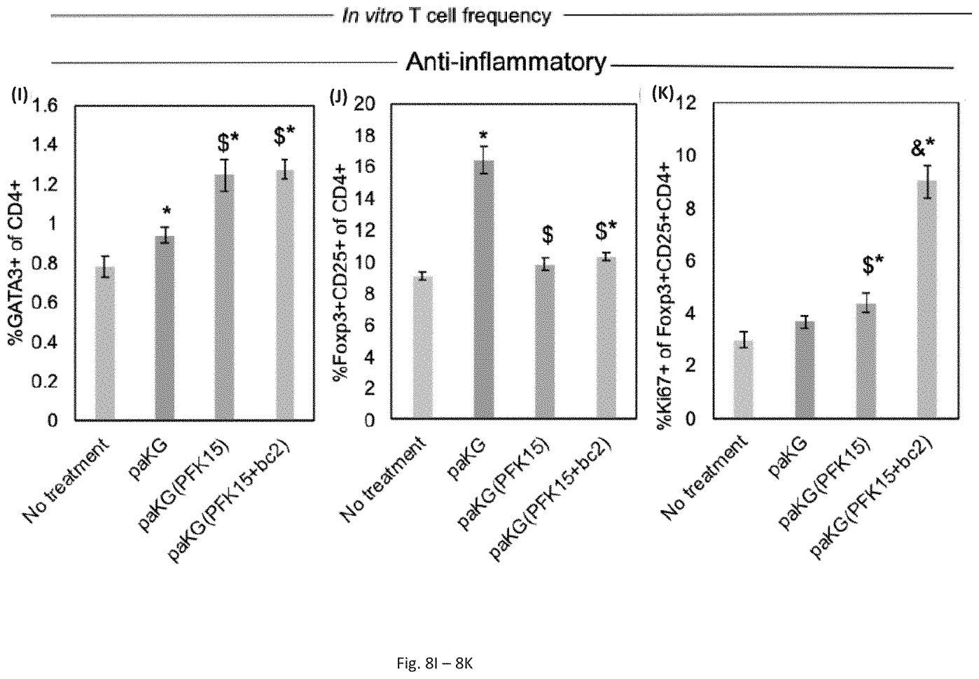

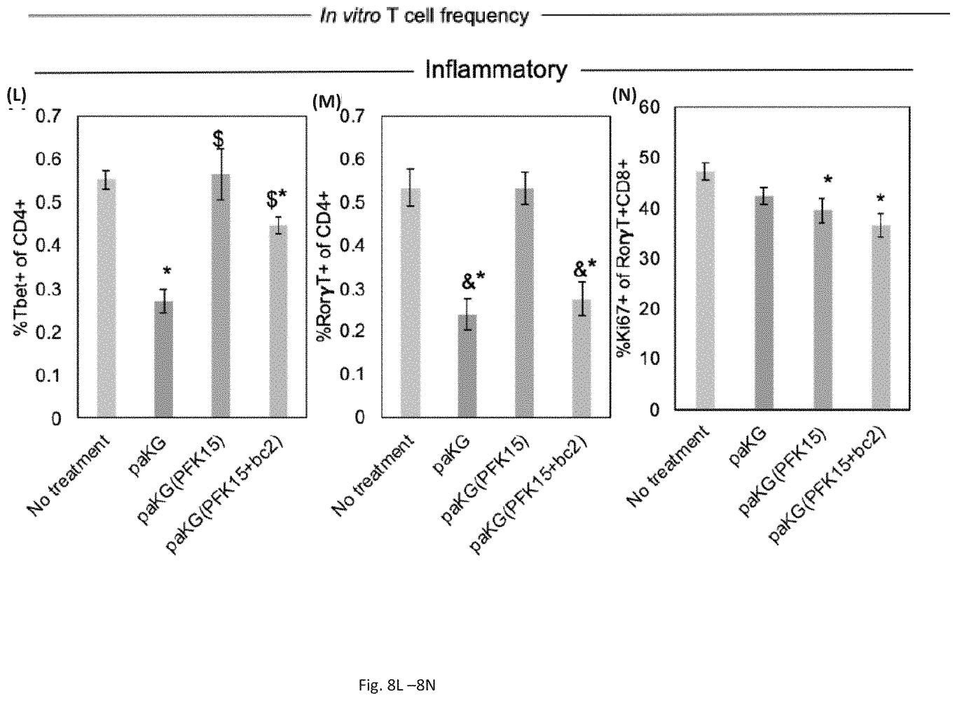

Since DCs indeed phagocytose paKG-based MPs in vivo, it was hypothesized that the observed reduction in paw swelling in the CIA mice treated with paKG(PFK15+bc2) MPs, may be due to the paKG(PFK15+bc2) MPs modulating DC function (FIG. 8A). Therefore, first effect of paKG(PFK15+bc2) MPs on in vitro cultured BMDCs in the form of altered CD86, MHCII, IL-10, IL-12p70 and TNFα expression in CD11c⁺ cells was tested. BMDC cultures were treated with the paKG(PFK15+bc2) MPs, and no treatment, paKG MPs, paKG(PFK15) MPs and LPS conditions were used as controls. It was observed that paKG MPs, paKG(PFK15) MPs and paKG(PFK15+bc2) MPs with LPS reduced CD86 expression in CD11c⁺ cells when compared to LPS alone condition (FIG. 8B, 8C). Specifically, paKG(PFK15+bc2) MPs with LPS had significantly decreased CD86 expression in CD11c⁺ cells by approximately 9-fold when compared to LPS alone (FIG. 8D). Moreover, BMDCs treated with paKG(PFK15+bc2) MPs, in the presence of LPS, nearly had a 40-fold decrease in CD86⁺MHCII⁺ BMDC populations when compared to CD86⁺MHCII⁺ BMDC populations in LPS alone (FIG. 8E). Furthermore, the fold increase of CD86⁻MHCII⁺ BMDCs to CD86⁺MHCII⁺ BMDCs in the paKG(PFK15+bc2) MP with LPS condition was significantly higher (˜23-fold) than the fold change in LPS alone, paKG+LPS (˜5-fold) and paKG(PFK15)+LPS (˜9-fold) (FIG. 8F).

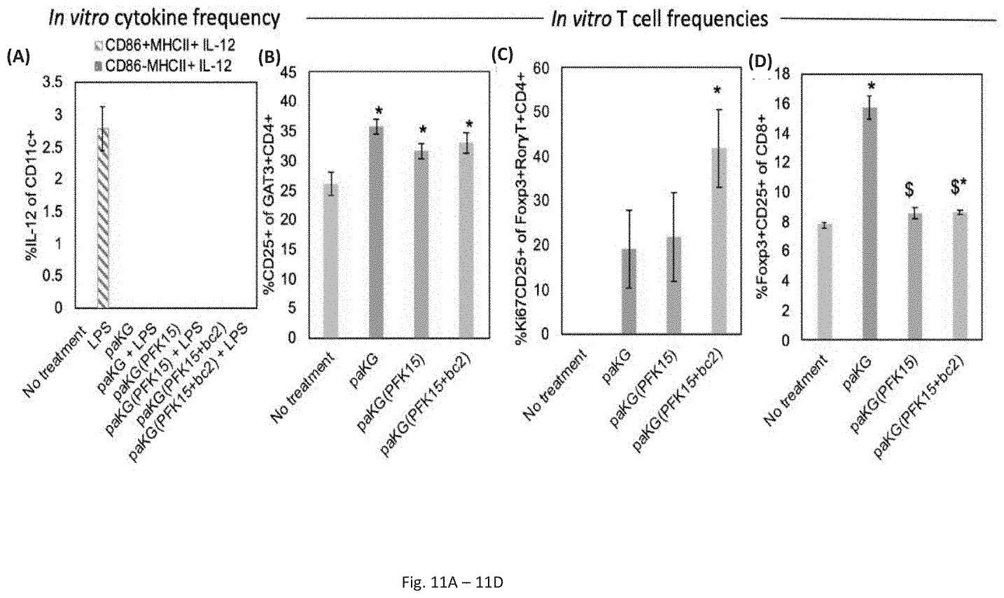

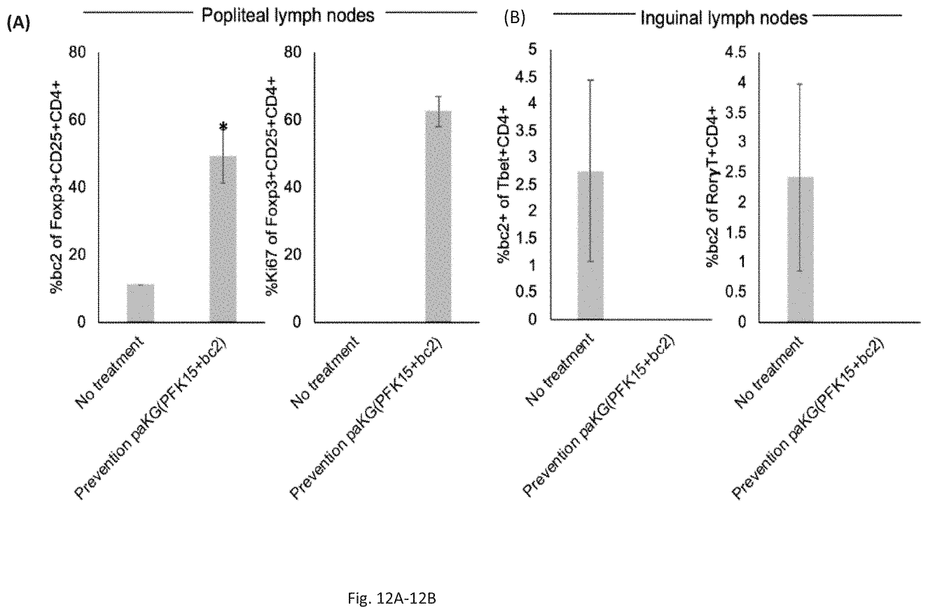

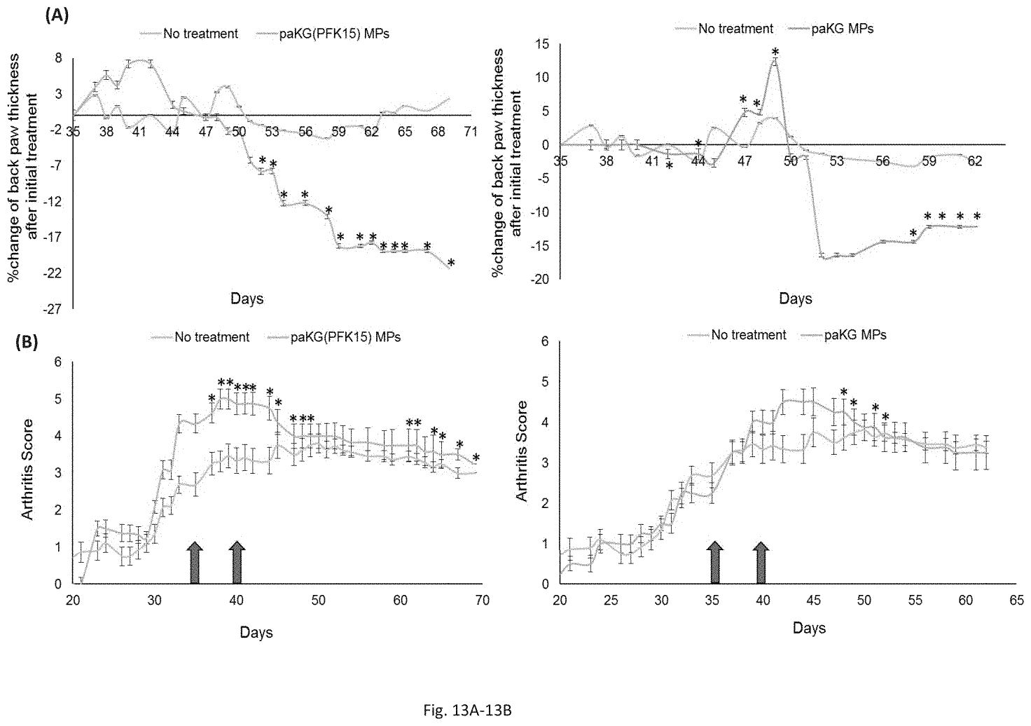

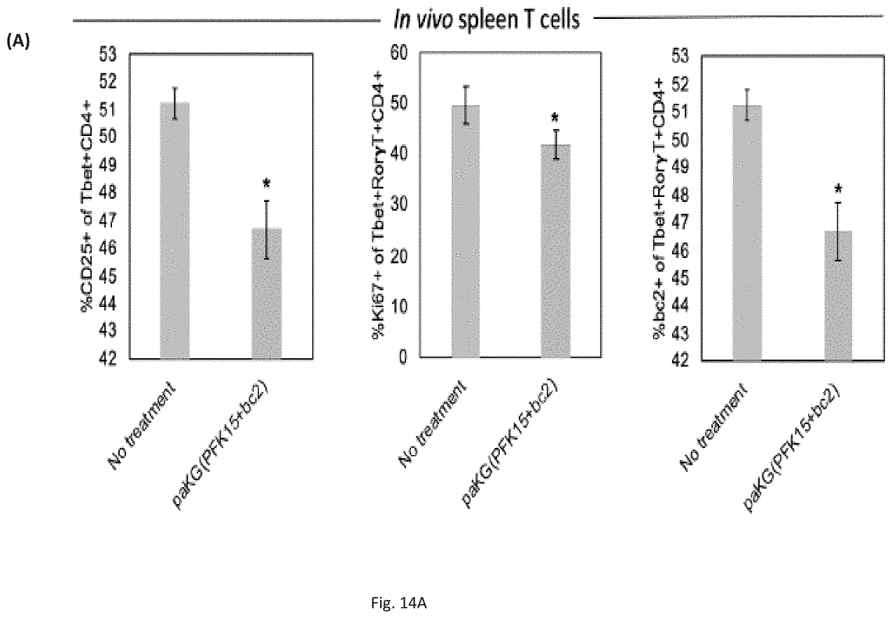

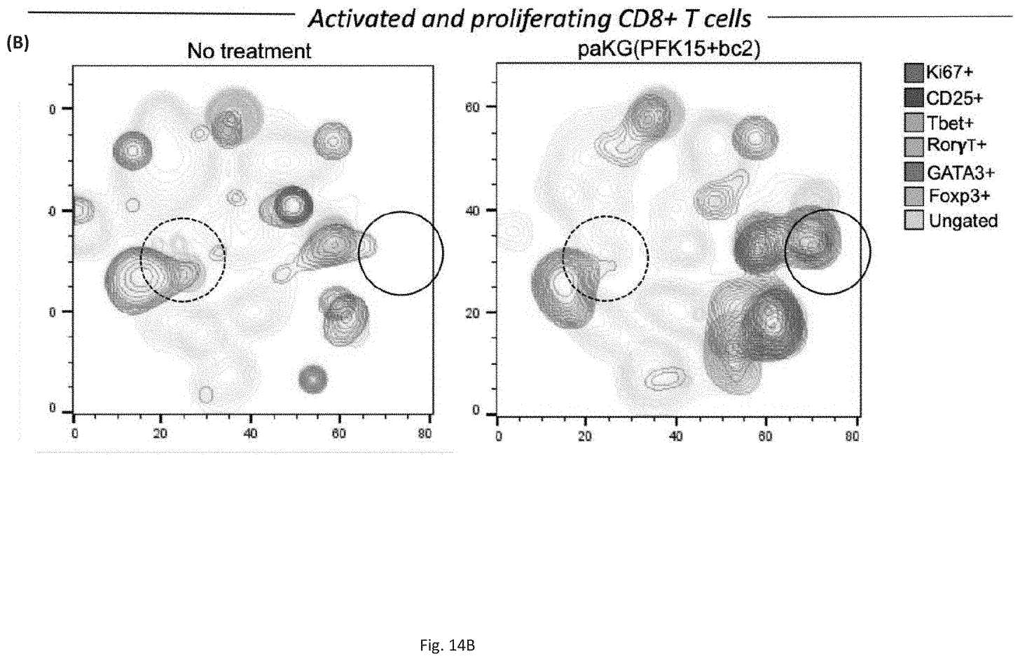

Notably, DCs can initiate adaptive immune responses by secreting signaling molecules, or cytokines, to communicate with themselves (autocrine) and to cells in their microenvironment (paracrine). Therefore, production of pro-inflammatory (IL-12p70 and TNFα) and anti-inflammatory (IL-10) cytokines were investigated in vitro. Although CD86⁻MHCII⁺IL-10⁺ and CD86⁺MHCII⁺IL-10⁺ production in the paKG(PFK15+bc2) MP treatment group with LPS was lower than LPS (FIG. 8G), TNFα production was significantly lower in the paKG(PFK15+bc2) MP treatment group with LPS in both CD86⁻MHCII⁺ and CD86⁺MHCII⁺ populations (FIG. 8H) and IL-12p70 production was only detectable in LPS alone in CD86⁺MHCII⁺ populations (FIG. 11A). Taken together, these data demonstrate that paKG-based MPs do not activate BMDCs and the combinatorial delivery of PFK15+bc2 via paKG MPs can reduce pro-inflammatory TNFα and IL-12p70 BMDC cytokine production.

paKG(PFK15+bc2) MPs Decrease CD86 Expression of CD11c⁺ Cells in the Injection Sites and Non-Draining Lymph Nodes of CIA Mice

Since in vitro DC studies demonstrated that paKG(PFK15+bc2) MPs can decrease CD86 expression in BMDCs and that paKG(PFK15) MPs can suppress pro-inflammatory gene expression, it was hypothesized that the paKG(PFK15+bc2) MPs can also modulate the expression of CD86 in CD11c⁺ cells in vivo in CIA mouse model. CIA induced mice were euthanized on day 70 and flow cytometry was performed on the treatment injection sites and the inguinal lymph nodes (located in the abdominal region) to understand if paKG(PFK15+bc2) MPs can impact CD11c⁺CD86⁺ expression in vivo. The LNs are essential to examine since LNs are crucial secondary lymphoid organs where DCs and T cells engage for the initiation of adaptive cell immunity. Therefore, if DCs are modulated in the LNs then this may lead to an eventual modulation in T cell responses as well. However, for this to occur, DCs would need to infiltrate the MP injection sites and traffic the MPs to the LNs. Therefore, the treatment injection sites were examined to assess if CD11c⁺ or F4/80⁺ (macrophage marker) cells infiltrate the injection sites in CIA mice. Since macrophages are professional phagocytes they were utilized here for comparison. When comparing the injection sites of mice treated with paKG(PFK15+bc2) MPs to the control of mice treated with paKG MPs, the paKG(PFK15+bc2) MP injection sites had an increase in CD11c (˜2-fold) and F4/80 (˜10-fold) expression. Moreover, the paKG(PFK15+bc2) MP injection sites had significantly lower percentages of CD86⁺CD11c⁺ expression, by approximately 8-fold, when compared to the paKG MP injection sites (FIG. 9C). These data suggest that CD11c⁺ and F4/80⁺ cells infiltrate the injection sites of paKG-based MPs in CIA mice, however, the delivery of PFK15+bc2 via paKG MPs drives the reduction in CD11c⁺CD86⁺ expression at the injection sites.

Next the ability of this particles to modulate CD86 expression in the draining LNs was tested. It was determined that the inguinal LNs had significantly lower CD86⁺ expression in CD11c⁺ cells as compared to the inguinal LNs of the no treatment control. Notably, MHCII expression in the in CD11c⁺ cells in paKG(PFK15+bc2) MP treatment group was not significantly different from no treatment. However, the inguinal LNs of the paKG(PFK15+bc2) MP treatment group had an increase in CD86⁻MHCII⁺ of CD11c⁺ DC populations and a significant decrease in CD86⁺MHCII⁺ of CD11c⁺ populations when compared to the no treatment inguinal LNs (FIG. 9B). Overall, these data indicate that the paKG(PFK15+bc2) MPs decrease the CD86 expression of CD11c⁺ cells that infiltrate the injection sites.

paKG(PFK15+bc2) MP Treated BMDCs Induce Suppressive T Cell Responses in an Allogeneic Mixed Lymphocyte Reaction In Vitro

Thus far, paKG(PFK15+bc2) MPs have shown to decrease CD86⁺MCHII⁺ of CD11c⁺ populations in vitro and in vivo, and since DCs play a central role in initiating and modulating T cell responses, the ability of paKG(PFK15+bc2) MP treated BMDCs to modulate T cell responses was assessed in vitro in a mixed lymphocyte reaction. As observed by flow cytometry, all paKG-based MP groups significantly increased the frequency of the anti-inflammatory cell type GATA3⁺CD4⁺ T_{H}2 as compared to no treatment. Notably, paKG(PFK15) MPs and paKG(PFK15+bc2) MPs had significantly increased T_{H}2 populations when compared to paKG MPs (FIG. 8I). Furthermore, as compared to no treatment, paKG and paKG(PFK15+bc2) MP conditions significantly upregulated the total population of anti-inflammatory Foxp3⁺CD25⁺CD4⁺ Treg frequencies. Although the frequency of Tregs was significantly higher in the paKG MP condition as compared to the paKG(PFK15+bc2) MP condition (less than 2-fold) (FIG. 8J), paKG(PFK15+bc2) MPs significantly increased proliferating (Ki67⁺) Treg populations, by ˜2-fold compared to paKG(PFK15) MPs. Moreover, paKG(PFK15) MPs also showed significantly increased proliferating (Ki67+) Treg populations as compared to no treatment. (FIG. 8K). Additionally, the paKG(PFK15+bc2) MPs also significantly increased anti-inflammatory cell frequencies of activated (CD25+) T_{H}2 cells and Foxp3⁺CD25⁺CD8⁺ T cells (FIG. 11B,11C). In all, these data indicate that BMDCs treated with paKG-based MPs increase anti-inflammatory T cell transcription factors.

Interestingly, when assessing pro-inflammatory T cell populations, it was determined that paKG and paKG(PFK15+bc2) MP conditions had a significantly decreased population of the Tbet⁺CD4⁺ T_{H}1 cell type. However, paKG MPs had significantly lower (less than ˜2-fold) T_{H}1 populations when compared to paKG(PFK15+bc2) MPs (FIG. 8L). Additionally, paKG and paKG(PFK15+bc2) MPs had significantly decreased frequencies of the RORγT⁺CD4⁺ T_{H}17 cell type, by approximately ˜2-fold, when compared to no treatment (FIG. 8M). Also, paKG(PFK15) and paKG(PFK15+bc2) MPs significantly downregulated populations of proliferating RORγT⁺CD8⁺ type 17 cytotoxic T cells (Tc17) cells when compared to no treatment (FIG. 8N). These data indicate that the BMDC treatment condition of paKG(PFK15+bc2) MPs consistently upregulated anti-inflammatory T cell responses when compared to no treatment. Moreover, these data demonstrate that paKG(PFK15+bc2) MP treated BMDCs can increase anti-inflammatory T cell transcription factors of Foxp3 and GATA3, while simultaneously decreasing pro-inflammatory T cell transcription factors of Tbet and RORγT. Furthermore, these data also strongly suggest that the modulation in adaptive T cell responses in vitro may stem from the induction of tolerogenic phenotypes in BMDCs that are treated with paKG(PFK15+bc2) MPs.

paKG MPs Delivering PFK15 and bc2 Induced Antigen-Specific T Cell Responses in Local and Systemic Sites of CIA Mice

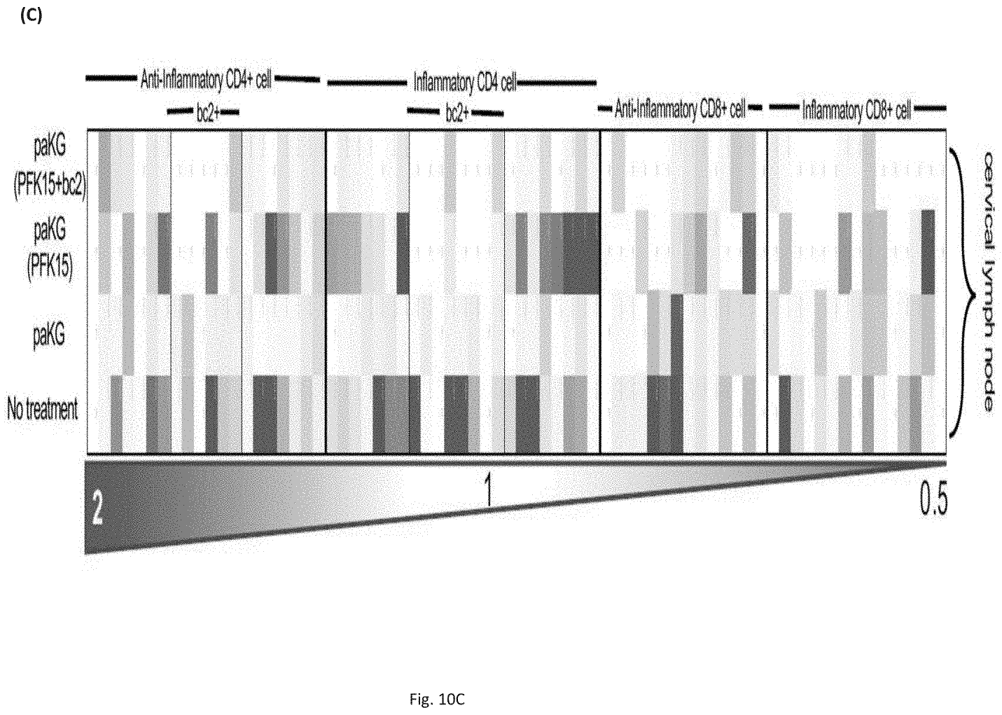

In order investigate if paKG(PFK15+bc2) MPs can induce antigen-specific T cell-mediated suppression in vivo, a CIA mouse model, where bc2 drives the onset and progression of RA, was utilized. After mice were euthanized on day 70, the draining popliteal lymph nodes (pLNs—near hind legs) and spleen were extracted and analyzed for different T cell phenotypes using flow cytometry. The pLNs are localized near the injection site and therefore were denoted as the local measurement of the treatment and the draining lymph nodes in this study. Whereas the spleen and cervical lymph nodes (cLNs—located in the neck) represented the systemic measurements of the treatment. Notably, the draining pLNs of paKG(PFK15+bc2) MP treated mice had significantly increased proliferating Treg populations as compared to no treatment. Additionally, mice receiving paKG(PFK15+bc2) MPs, when compared to no treatment, had significantly decreased T_{H}1, T_{C}17 and proliferating and activated CD8⁺Foxp3⁺RORγT⁺ (˜8-fold) populations in the pLNs. Importantly, paKG(PFK15+bc2) MP treatment groups showed significantly higher percentages of proliferating antigen-specific Treg populations (˜2-fold) and significantly decreased percentages of activated antigen-specific T_{H}17 cell types in the pLNs when compared to no treatment (FIG. 10A). These data indicate that CIA mice treated with paKG(PFK15+bc2) MPs can increase anti-inflammatory responses and decrease inflammatory T cell responses in local sites. Importantly, paKG(PFK15+bc2) MP treated mice can generate antigen-specific immune suppression in the draining pLNs.

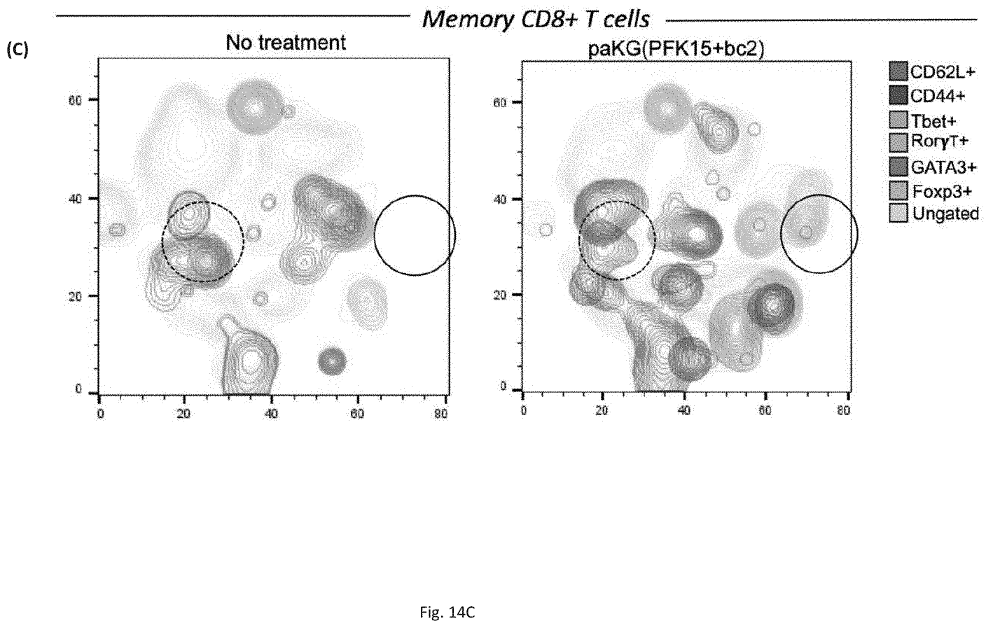

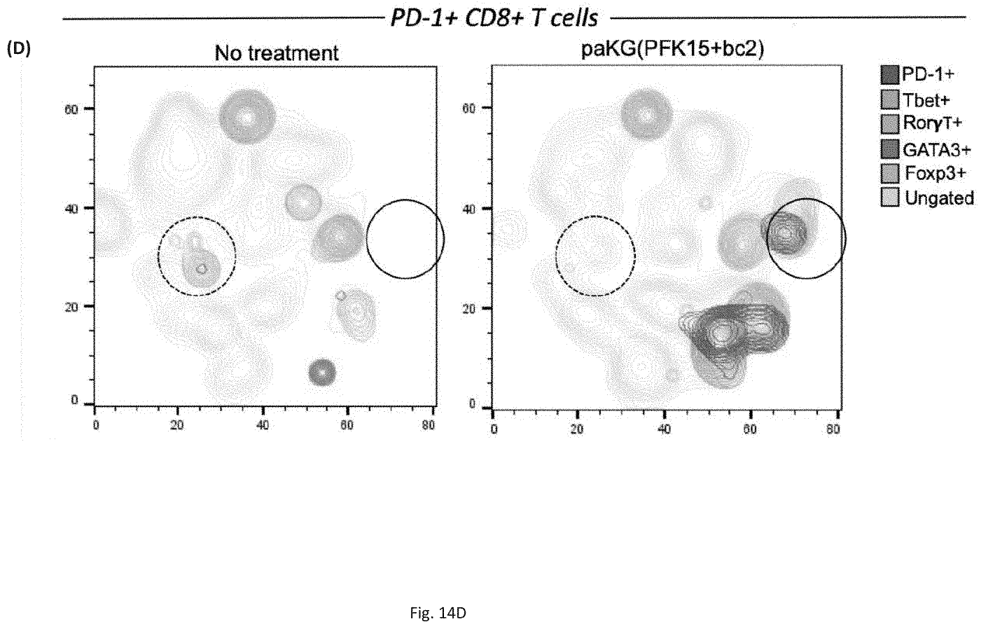

Furthermore, when looking at cytotoxic CD8⁺ tSNE plots within the draining pLNs, the paKG(PFK15+bc2) MP group revealed new populations when being compared to the no treatment group. CD8⁺ T cells are implicated in autoimmunity and, therefore are important cells especially when considering their ability to directly destroy their targets. However, similar to T helper cells, cytotoxic T cells have pro- and anti-inflammatory phenotypes as well. Interestingly, CD25 and Ki67 markers appeared in the anti-inflammatory Foxp3⁺CD25⁺CD8⁺ and Tc2 populations of paKG(PFK15+bc2) MP treated mice (FIG. 14B). Similarly, memory CD44 and CD62L were detected in Foxp3⁺CD25⁺CD8⁺ and T_{C}2 populations in paKG(PFK15+bc2) MP treated mice. These observations were not found in the no treatment mice (FIG. 14C).

Notably, when assessing the systemic T cell effects of paKG(PFK15+bc2) MPs in the spleen, it was observed that paKG(PFK15+bc2) MP treated mice had significantly decreased inflammatory T cell populations of activated T_{H}1, activated T_{H}17 and proliferating CD4⁺Tbet⁺RORγT⁺ populations as compared to the no treatment control. Significantly decreased frequencies of antigen-specific CD4⁺Tbet⁺RORγT⁺ populations were also detected in the spleen of paKG(PFK15+bc2) MP treated mice when compared to no treatment (FIG. 14A). Overall, these data strongly suggest that the delivery of PFK15+bc2 via paKG MPs can generate immunosuppressive antigen-specific T cell responses in vivo. Specifically, the T cell phenotype of CIA mice treated with paKG(PFK15+bc2) MPs appears to be skewed toward bc2-specific anti-inflammatory responses as observed by an up-regulation of bc2-specific Treg populations and down regulation of pro-inflammatory bc2-specific T_{H}1 and T_{H}17 responses, which may potentially be due to the modulation of DC responses in the lymph nodes. Notably, antigen-specific T cell responses were detected in the local draining pLNs and systemically, as determined by the spleen.

In order to confirm if the observed antigen-specific T cell responses in the CIA mouse model were indeed systemic, ex vivo recall reactions were performed on non-draining cervical LNs (cLNs). After the CIA mice were euthanized on day 70, cells from the non-draining cLNs were isolated from paKG(PFK15+bc2) MP treated mice and the control mice (no treatment, paKG MPs and paKG(PFK15) MPs and paKG(PFK15+bc2) MPs), and these cells were then treated with bc2 antigen or bovine serum albumin (BSA). A heatmap was utilized to qualitatively depict if T cells exposed to bc2 proliferated differentially as compared to T cells exposed to BSA (scale of fold change in proliferation is shown—FIG. 10C). Overall, a lower frequency in pro-inflammatory CD4⁺ and CD8⁺ T cells was observed in paKG(PFK15+bc2) MP treated mice as compared to control mice. Importantly, the paKG(PFK15+bc2) MP treatment group displayed lower bc2-specific pro-inflammatory CD4⁺ T cell populations as compared to no treatment (FIG. 10C). Specifically, it was observed that the frequency of activated T_{H}1 cells was significantly decreased in mice treated with paKG(PFK15+bc2) MPs as compared to the no treatment control. Proliferating T_{H}2 cells (˜2-fold) and proliferating Foxp3+CD25+CD8+(˜3-fold) were significantly higher in the paKG(PFK15+bc2) MP treatment group, however, this difference was not observed in the no treatment mice (FIG. 10B). Taken together, these data strongly suggest that T cells in the paKG(PFK15+bc2) treatment group are capable of generating immunosuppressive antigen-specific T cell responses in systemic sites when introduced to the self-antigen. Importantly, this data also indicates that the significant shift toward immunosuppressive responses may potentially be due to the generation of proliferating antigen specific Tregs.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a polymer of α-ketoglutarate for the treatment of a disease or disorder.

2. The composition of claim 1, wherein the polymer is a polymeric particle.

3. The composition of claim 1 further comprising at least one additional agent.

4. The composition of claim 3, wherein the at least one additional agent is selected from the group consisting of a peptide, a protein, an antibody, a nucleic acid molecule, a small molecule chemical compound, and any combination thereof.

5. The composition of claim 3, wherein the at least one additional agent is selected from the group consisting of PFK15, a connective tissue antigen, bc2, Cb-839, TRP2 and a combination thereof.

6. The composition of claim 5, wherein the composition comprises an α-ketoglutarate polymeric particle comprising PFK15, bc2, or a combination thereof.

7. (canceled)

8. (canceled)

9. The composition of claim 1, wherein the composition is a pharmaceutical composition.

10. A method of suppressing an immune response in a subject in need thereof, the method comprising administering the composition of claim 1 to the subject.

11. The method of claim 10, wherein the method comprises generating an immunosuppressive dendritic cell (DC) in the subject.

12. The method of claim 11, wherein the DC comprises a phenotype selected from the group consisting of MHCII⁺CD86^{Lo}IL-10⁺IL-12p70^{Lo} and MHCII⁺CD86^{Lo}IL-10⁺IL-12p70^{Lo}TNFα^{Lo}.

13. The method of claim 10, wherein the method comprises generating and antigen-specific immunosuppressive T cell in the subject.

14. The method of claim 13, wherein the T cell is selected from the group consisting of an antigen-specific regulatory T cell, antigen-specific T-helper 1, antigen-specific T-helper 17, antigen-specific T-helper 2 and antigen-specific T follicular helper cell.

15. A method of treating a disease or disorder in a subject in need thereof, the method comprising administering a composition of claim 1 to the subject.

16. The method of claim 15, wherein the disease or disorder is an autoimmune disease.

17. The method of claim 16, wherein the disease or disorder is rheumatoid arthritis (RA).

18. A method of modulating a metabolic pathway, the method comprising contacting a cell with the composition of claim 1.

19. The method of claim 18, wherein the metabolic pathway is glycolysis or the glutaminase pathway.

20. The method of claim 18, wherein the cell is an immune cell.

21. The method of claim 20, wherein the cell is a DC.

22. A kit for generating an immunosuppressive immune cell comprising a composition of claim 1.
